(19) United States
(12) Patent Application Publication
(10) Pub. No.: US 2007/0196335 A1
(43) Pub. Date: Aug. 23, 2007

(54) Immune Modulation By Regulating Expression Of The "Minor" Gene In Immune Dendritic Cells

(75) Inventors: Drew Pardoll, Brookeville, MD (US); Katharine A. Whartenby, Baltimore, MD (US); Kevin S. Gorski, St. Paul, MN (US); Camie Chan, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 10/588,888

(22) PCT Filed: Feb. 9, 2005
(86) PCT No.: PCT/US05/04060

§ 371 Date: Apr. 6, 2007

Related U.S. Application Data
(60) Provisional application No. 60/542,987, filed on Feb. 9, 2004.
(60) Provisional application No. 60/561,417, filed on Apr. 12, 2004.

(51) Int. Cl. A61K 48/00; C12N 5/08

(52) U.S. Cl. 424/93.2; 514/44; 435/372; 424/93.21

(57) ABSTRACT

Mitogen induced nuclear orphan receptor (MINOR) is described as inducing apoptosis in dendritic cells (DCs). Downregulation of its expression results in a downregulation of apoptosis. A novel approach of inhibiting DC apoptosis is described employing small interfering RNA (siRNA) that targets MINOR. Improved DC-based vaccines exhibiting longer lifespan of DCs, increased potency of DCs, and enhanced immunogenicity are described.

### CROSS REFERENCE TO RELATED APPLICATIONS

This application claims priority to U.S. Provisional Patent Application Ser. No. 60/542,987 and 60/561,417 filed Feb. 9, 2004 and Apr. 12, 2004, respectively, the entire disclosures of which are incorporated herein by reference in its entirety.

### FIELD OF THE INVENTION

The present invention relates to the modulation of a targeted gene in certain immune cells. This modulation can result in the inhibition or stimulation of certain immune response processes.

### BACKGROUND OF THE INVENTION

Dendritic cells (DCs) are scarce cells in the immune system that are specialized in processing antigens, presenting to naive T lymphocytes, and playing an important role in initiating host immune responses. DCs also play an important role in maintaining tolerance. Studies using labeled DCs suggest that they are replaced every 3-4 days (Asavaroengchai, W., Kotera, Y. & Mule, J. J., Proc Natl Acad Sci U S A 99, 931-6, 2002), implying a limited window during which T cells can encounter DCs presenting antigens. While many advances have been made in understanding the nature of antigen processing and presentation, regulation of DC lifespan has not been well explored.

Apoptosis is a highly complex process that allows a cell to commit suicide in a controlled manner. Death receptors belonging to the tumor necrosis factor receptor superfamily can transmit apoptotic signals initiated by their specific ligands. One central component of the apoptotic machinery is a family of proteases, the caspases, which participate in a cascade of proteolytic cleavages, finally resulting in the death of the cell. Of note, a number of reports have appeared describing a caspase-independent form of apoptosis.

Previous studies have shown that manipulation of the bcl-2 pathway led to inhibition of apoptotic cell death of DCs. DCs were shown to downregulate the anti-apoptotic bcl-2 upon maturation, leading to their progression to cell death. Mice were generated that were transgenic for bcl-2, and that had increased numbers of DCs. Further, DCs from these transgenic mice as vaccines generated an enhanced immune response. (Nopora, A. & Brocker, T., J Immunol 169, 3006-14, 2002). Also, while tumors can induce apoptosis of DCs, transduction of ex vivo-generated DCs with the anti-apoptotic Bcl-xl increased their resistance to apoptosis and improved tumor vaccine efficacy. (Pirtskhalaishvili, G. et al., J Immunol 165, 1956-62, 2000)

Nurr77 (NGFI-γ or TR3), an orphan member of the steroid/thyroid/retinoid nuclear receptor superfamily (Kastner et al., 1995; Mangelsdorf and Evans, 1995; Zhang, 2002), plays roles in regulating growth and apoptosis (Winoto and Littman, 2002; Zamzami and Kroemer, 2001; Zhang 2002) in T lymphocytes. The Nurr77 family members, Nur77, Nor-1, and Nurr1, have the typical steroid receptor organization composed of an N-terminal transactivation domain, a central DNA-binding domain containing two zinc fingers, and a C-terminus with homology to hormone-binding domains (Carson et al., 1990). Recent studies indicate that Nur77 translocates from the nucleus to the mitochondria, where it directly exerts its pro-apoptic effects.

The role of Nur77 in T cell development has been fairly well documented. The actual mechanism of cell death induction by these molecules is not as clear. Nur77 is a nuclear, DNA binding, transcriptional regulator, so the most likely mechanism is via transcription of downstream effectors (Kuang, A. A., Cado, D. & Winoto, A., Eur J Immunol 29, 3722-8, 1999). However, other evidence suggests that Nur77 can mediate its apoptotic affect in the mitochondria (Brenner, C. & Kroemer, G., Science 289, 1150-1, 2000).

Mouse MINOR belongs to the Nur77 family of orphan receptors that includes Nur77 and the rat homologue of MINOR, Nor 1, which is involved in activation induced cell death of T cells (Cheng, L. E., Chan, F. K., Cado, D. & Winoto, A., Embo J 16, 1865-75, 1997; Liu, Z. G., Smith, S. W., McLaughlin, K. A., Schwartz, L. M. & Osborne, B. A., Nature 367, 281-4, 1994). and also caspase-independent cell death of macrophages. (Kim, S. O., Ono, K., Tobias, P. S. & Han, J., J Exp. Med 197, 1441-52, 2003). The role of Nur77 in T cell development has been fairly well documented. Interestingly, Nor-1 appears to be functionally redundant with Nur77 not only in DNA binding specificity and ability to transactivate from the NBRE promoter, but also in its ability to induce apoptosis in T cells. Furthermore, a dominant negative Nur77 gene fragment lacking the transactivation domain blocks the induction of apoptosis by both Nur77 and Nor-1. In addition, transgenic mice expressing the rat MINOR homologue, under the control of the lck promoter, exhibited a 15-fold reduction in their thymocyte number, clearly showing the impact of this pathway on T cells (Cheng, L. E., Chan, F. K., Cado, D. & Winoto, A., Embo J 16, 1865-75, 1997). However, the notion that Nor-1 has been shown to bind and activate transcription from the NBRE indicates that it is likely capable of activating the same genes that are activated by other members of this orphan receptor family. Indeed, Nor-1 has been shown to express in activated T cells and demonstrated for its role in T cell receptor-mediated apoptosis in vivo.

Improving vaccination strategies for tumors is a significant goal of immunotherapy. As a result of the potency of dendritic cells (DCs) as antigen presenting cells (APCs), DCs have been investigated for both their biology and potential as therapeutic agents. While many advances have occurred in this field, highly potent and durable anti-tumor immune responses have been difficult to achieve through these vaccines. It now appears that DC vaccines can elicit strong immune responses, but they are limited, in part, by their short lifespan in vivo. While much emphasis has been placed on studying antigen (Ag) uptake, processing, and presentation, as well as co-stimulatory signal delivery by DCs, little is known about regulation of DC lifespan.

DC-based vaccines have been tested in a number of animal models, and have also been translated to clinical medicine in several trials. Thus, the hope for successful immunotherapy through this mechanism is high. To produce DC vaccines, there are two primary methods: First, precursor cells can be isolated, grown ex vivo and differentiated in culture, then subsequently re-infused; second, they can be generated in vivo through the systemic administration of GM-CSF and/or Flt-3 ligand (FLT3L). While these approaches have led to some degree of tumor immunity, they have also exhibited limitations (Borges, L. et al., J Immunol 163, 1289-97, 1999). The identification of tumor-specific and tumor-associated Ags has led to therapies such as vaccination with recombinant viruses or DCs modified to express Ags, but these have also had limited effects. (Marshall, J. L. et al., J Clin Oncol 18, 3964-73, 2000; Morse, M. A. et al., Cancer Invest 21, 341-9, 2003; Ridgway, D., Cancer Invest 21, 873-86, 2003).

Therapeutic cancer vaccination depends on effective transfer of Ag to DCs and trafficking of the DCs to the secondary lymphoid organs. While many clinical trials have been initiated with ex vivo-generated DCs, for the most part, no long-term cures have been achieved. Of note, DCs are more resistant to some apoptotic pathways than other cells as a result of expression of molecules such as FLICE inhibitory protein (cFLIP), which can block caspase 8 activation and the subsequent apoptotic cascade (Leverkus, M. et al., Blood 96, 2628-31, 2000). Additionally, signaling by TRANCE, or CD154 have been shown to prevent apoptosis in DCs (McLellan, A. et al., Eur J Immunol 30, 2612-9, 2000; Wong, B. R. et al., J Exp Med 186, 2075-80, 1997). In spite of these, the DCs generally have a very short lifespan in vivo. In fact, animal models and clinical trials suggest that one major issue with the ex vivo expansion and loading of DCs followed by re-injection is that relatively few DCs successfully traffic to spleen or lymph nodes (Eggert, A. A. et al., Cancer Res 59, 3340-5, 1999), and those that do are rapidly cleared by host CTL (Cayeux, S. et al., Eur J Immunol 29, 225-34, 1999). In addition, NK cells can kill DCs through TRAIL-mediated apoptosis (Hayakawa, Y. et al., J Immunol 172, 123-9, 2004), and further studies using labeled DCs suggest that the cells are replaced every 3-4 days (Kamath, A. T., Henri, S., Battye, F., Tough, D. F. & Shortman, K., Blood 100, 1734-41, 2002), implying a limited window during which T cells can encounter DCs presenting Ag. Consequently, it would be highly desirable if methods were available to increase the lifespan of DCs and maximize their effect in a number of immune processes involving Ag presentation, including vaccinations.

### SUMMARY OF THE INVENTION

Recent investigation of some of the genes that are upregulated in DCs has led to the identification of one that appears to play a significant role in limiting the lifespan of DCs, mitogen induced nuclear orphan receptor (MINOR). The subtractive hybridization analysis between activated macrophages and DCs that was employed to identify selectively expressed genes revealed a highly upregulated expression of the mouse homolog to human MINOR in mature DCs.

Extending the longevity of DCs allows for their improved immunogenicity. In one embodiment, the present invention is directed to a method for substantially inhibiting apoptosis in dendritic cells comprising the prevention or inhibition of the expression of MINOR in said cells. An inhibition of apoptosis constitutes an experimentally quantifiable level of inhibition, as shown in the Examples, below. A prevention or inhibition of the expression of MINOR constitutes a downregulation of the expression of MINOR great enough to result in some measurable degree of inhibition of apoptosis in the DCs.

In another embodiment of the invention, the expression of MINOR in DCs is prevented or inhibited by the transduction of the cells with a lentiviral vector encoding an siRNA construct having substantial sequence homology to MINOR. Substantial sequence homology constitutes about 60% or greater sequence homology, preferably about 75% or greater, more preferably about 85% or greater. The sequence homology must be great enough to allow effective targeting of MINOR by the siRNA construct.

In yet another embodiment of the invention, the dendritic cells are bone marrow dendritic cells.

In one embodiment, the invention is directed to a method for improving the survival time after infusion of ex vivo-generated dendritic cells, said method comprising transducing said cells with a lentiviral vector encoding an siRNA construct having substantial sequence homology to MINOR or through additional means of introducing a MINOR-blocking reagent, e.g., with a plasmid construct through transfection by any number of standard methods, and infusing the transduced cells into a mammalian subject. In a further embodiment of the invention, the mammalian subject is human.

In one embodiment, the invention is directed to enhancing the immunogenicity of dendritic cells, said method comprising transducing said cells with a lentiviral vector encoding an siRNA construct having substantial sequence homology to MINOR.

In another embodiment, the invention is directed to a method for enhancing the capacity for dendritic cells to stimulate tolerant T cells, said method comprising transducing said cells with a lentiviral vector encoding an siRNA construct having substantial sequence homology to MINOR.

In one embodiment, the invention is directed to a dendritic cell-based vaccine comprising siRNA having substantial sequence homology to MINOR. In another embodiment of the invention, the vaccine is for cancer, viral disease, bacterial disease, or immune disorders.

In one embodiment, the invention is directed to a method for preparing a DC-based vaccine, comprising the step of preparing an siRNA construct having substantial sequence homology to MINOR to target MINOR on a molecular level.

In another embodiment, the invention is directed to a method of preserving the CD11c+population of dendritic cells, comprising transducing hematopoietic stem-progenitor cells with a lentiviral vector encoding an siRNA construct having substantial sequence homology to MINOR.

In an additional embodiment, the invention is directed to a method for stably decreasing or substantially suppressing the expression of MINOR in dendritic cells, said method comprising the steps of transducing hematopoietic stem-progenitor cells with a vector encoding an siRNA construct having substantial sequence homology to MINOR and transplanting the transduced cells into a myeloablatively treated mammalian subject.

Specifically in one embodiment of the present invention a dendritic cell is transduced with a construct containing the a small interfering RNA comprising the double stranded nucleotide sequence of 5′GATCCCCTGCCCTTGTCCGAGCTTTATTCAAGAGATAAAGCTCGGACAAGGGCATTTTTGGAAA-3′; forward and 5′AGCTTTTCCAAAAATGCCCTTGTCCGAGCTTTATCTCTTGAATAAAGCTCGGACAAGGGCAGGG-3′; reverse.

In additional embodiments, the invention is directed to improved DC vaccines.

Thus, the present invention utilizes the discovery of MINOR as a significant gene with regard to basic DC function in that it may help regulate DC lifespan, thereby limiting uncontrolled T cell activation and also thereby serving as a target for improving DC-based therapies.

### BRIEF DESCRIPTION OF THE FIGURES

FIG. 1 illustrates the results of qPCR analysis of relative MINOR expression in vitro in DCs and activated macrophages; Lane 1 corresponds to activated macrophages; Lane 2 corresponds to day 4 cultures of bone marrow DCs; Lane 3 corresponds to day 8 cultures of bone marrow DCs; Lane 4 corresponds to myeloid CD11c hi DCs isolated from Balb/c lymph nodes.

FIG. 2 schematically depicts the structure of MINOR and illustrates the results of protein sequence alignment of MINOR with other members of the Nur77/Nurr1 steroid hormone receptor family, showing % identity to the most similar sequences; the subfamilies of this gene are likewise shown.

FIG. 3 represents an immunoblot depicting MINOR expression in mouse BMDCs at the protein level.

FIG. 4 depicts the expression of MINOR and Nurr77 in various cell (primarily dendritic cell) populations, respectively, in bar graph form.

FIG. 5 illustrates cell death as measured via FACS analysis of the DC-like cell line DC 2.4 transfected with the rat homolog MINOR cDNA or pEGFP-N2. Cells were gated on GFP+ populations in order to specifically compare transduced cells.

FIG. 6 illustrates in bar graph form the results of quantitative PCR analysis of MINOR expression by bone marrow progenitor cell-derived DCs transduced with siRNA-MINOR (as compared with unmodified mature DCs).

FIG. 7 illustrates the FACS results of staining for apoptosis in DC2.4 cells transduced with LV-siRNA-MINOR or LV-control-siRNA and then transfected with rat Nor-1; transduced DC2.4 cells were gated based on GFP fluorescence (GFP+) and compared with the non-transduced DC2.4 (GFP negative).

FIG. 8 illustrates the FACS results of staining for apoptosis in BMDCs transduced with LV-siRNA-MINOR-GFP or LI-control-siRNA-GFP. The FACS plots are gated either on GFP⁻ (right) or GFP+ (left) for either siRNA-MINOR or. control vs 7-AAD (indicating the dead population in the upper right quadrant); the GFP⁻ fractions in both populations represent the untransduced groups, while the GFP⁺ populations are transduced; the control GFP⁺ populations express a control siRNA-GFP while the siRNA-GFP population expresses both GFP and siRNA-MINOR.

FIG. 9 illustrates the results of monitoring the survival of ex vivo generated BALB/c BMDCs transduced with siRNA-MINOR-GFP or control GFP and injected into mice; the histograms shown are gated on CD11c+ cells for this analysis.

FIG. 10 depicts, in bar graph, FACS plot, and point distribution graph form, T cell expansion, as measured in terms of staining for 6.5 and CD4, observed in mouse spleen and lymph nodes after transfer of 6.5 BMDCs transduced with siRNA-MINOR-GFP or control-siRNA-GFP and pulsed with class II restricted peptide for HA into the mice.

FIGS. 11a and 11b illustrate, in FACS plot and distribution graph form, respectively, the FACS results of staining with antibodies for thy 1.2 and CD4 in ex vivo generated BMDCs expressing HA as self antigen (“142”), transduced with si-RNA MINOR or siRNA control and pulsed with HA class II peptide prior to subcutaneous injection into mice; unpulsed DCs were included as a control.

FIG. 12 illustrates, in bar graph form, the relative in vivo expression (in BALB.c mice) of MINOR upon systemic DC activation, in comparison with that measured for naïve DCs.

FIG. 13 illustrates, in bar graph form, the relative in vivo expression of MINOR in mice transplanted with siRNA-MINOR- or siRNA control-transduced HSCs and allowed to engraft; the lanes are as follows: (1) plasmacytoid (p)DCs from the spleen of the 1203 transplants; (2) control spleen pDCs; (3) LN pDCs from the 1203 transplants, and (4) LN pDCs from the control transplants; values were normalized to actin, and the lowest value was arbitrarily set to 1.

FIG. 14 illustrates the relative expression of siRNA-MINOR vs. control siRNA in CD11c+ cells vs. CD11c− cells; for the FACS analysis, the cells were stained for CD11c and 7-AAD; shown are representative plots of LN for GFP by CD11c, in order to compare relative expression of the vectors in DC (upper 2 quads) vs non-DC (lower 2 quads) populations; the bar graph shows the average and SD of 6 mice/group.

FIG. 15 illustrates the FACS results for an analysis of expression of siRNA-MINOR vs. siRNA-control based on CD86 expression; shown are representative FACS plots for siRNA-MINOR, left, and the control, right, for CD86 by GFP.

FIG. 16 illustrates the results of an analysis of the viability of the CD11c+ DC progeny of HSCs transduced with siRNA-MINOR vs. siRNA-control prior to transplant; Separate comparisons were made within each group of mice (control GFP⁺ vs control GFP⁻ and siRNA-MINOR GFP⁺ vs siRNA-MINOR GFP⁺).

FIG. 17 illustrates the relative expression (normalized to actin) of human MINOR in: CD34⁺ hematopoietic cell progenitors, LPS activated-monocyte derived-macrophages, and activated dendritic cells.

FIG. 18 depicts the nucleotide sequence for MINOR (SEQ ID NO. 1).

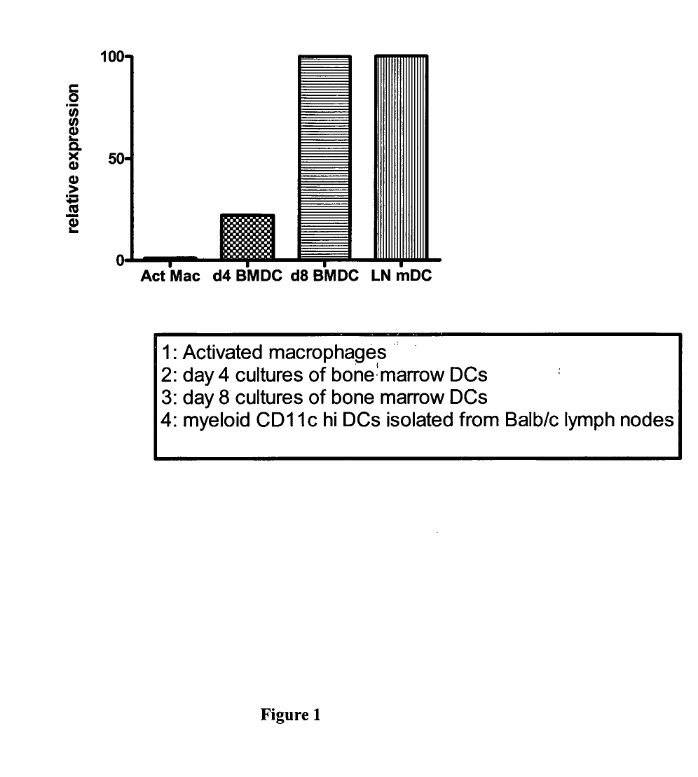

### DETAILED DESCRIPTION OF THE INVENTION

Improving vaccination strategies for tumors is a significant goal of immunotherapy. As a result of the potency of dendritic cells (DCs) as antigen presenting cells (APCs), DCs have been investigated for both their biology and potential as therapeutic agents. While many advances have occurred in this field, highly potent and durable anti-tumor immune responses have been difficult to achieve through these vaccines. It now appears that DC vaccines can elicit strong immune responses, but they are limited, in part by their short lifespans in vivo. While much emphasis has been placed in studying antigen (Ag) uptake, processing and presentation as well as costimulatory signal delivery by DCs, little is known about regulation of DC lifespan. We have identified one gene that appears to play a significant role in limiting the lifespan of DCs, mitogen induced nuclear orphan receptor (MINOR). The identification of this gene was made through a subtractive hybridization analysis between activated macrophages and DCs, which revealed a highly upregulated expression of the mouse homolog to human MINOR in mature DCs. Many studies have now focused on the features of DCs that allow them to be such potent APCs, including analyses of signals for activation that are important for DC function (such as co-stimulatory molecules, molecules involved in Ag processing and presentation, etc.). Once DCs have been activated, however, they are thought to have a relatively short lifespan in vivo, which presumably serves to limit clonal expansion in an immune response. Thus, it appears that DCs are messengers with a limited time to carry their Ag to secondary lymphoid organs and activate T cells in the context of a pro-inflammatory environment.

Minor Gene and its Protein Product

MINOR was identified as a gene that is highly upregulated and selectively expressed in DCs. This gene belongs to the Nur77 family of orphan receptors that includes Nur77 and the rat homologue of MINOR, Nor 1, which is involved in activation induced cell death of T cells (Cheng, L. E., Chan, F. K., Cado, D. & Winoto, A., Embo J 16, 1865-75, 1997; Liu, Z. G., Smith, S. W., McLaughlin, K. A., Schwartz, L. M. & Osborne, B. A., Nature 367, 281-4, 1994) and also caspase-independent cell death of macrophages (Kim, S. O., Ono, K., Tobias, P. S. & Han, J., J. Exp. Med 197, 1441-52, 2003). The role of Nur77 in T cell development has been fairly well documented. Interestingly, Nor-1, appears to be functionally redundant with Nur77 not only in DNA binding specificity and ability to transactivate from the NBRE promoter, but also in its ability to induce apoptosis in T cells. Furthermore, a dominant negative Nur77 gene fragment lacking the transactivation domain blocks the induction of apoptosis by both Nur77 and Nor-1. In addition, transgenic mice expressing the rat MINOR homologue, under the control of the lck promoter, exhibited a 15 fold reduction in their thymocyte number, clearly showing the impact of this pathway on T cells (Cheng, L. E., Chan, F. K., Cado, D. & Winoto, A., Embo J 16, 1865-75, 1997). Previous results show that Nur77 is not upregulated as DCs mature thus we are hypothesizing that the mouse MINOR substitutes for the signal to induce apoptosis, in a selective fashion.

The actual mechanism of cell death induction by these molecules is not as clear. Nur77 is a nuclear, DNA binding, transcriptional regulator, so the most likely mechanism is via transcription of downstream effectors (Kuang, A. A., Cado, D. & Winoto, A., Eur J Immunol 29, 3722-8, 1999). However, other evidence suggests that Nur77 can mediate its apoptotic effect in the mitochondria (Brenner, C. & Kroemer, G., Science 289, 1150-1, 2000). Using a GFP tagged Nur77 to follow its movement through cells, a translocation from the nucleus to mitochondria was observed with concomitant cytochrome c release in the presence of apoptosis inducing stimuli in these studies. The mitochondrial localization of GFP-Nur77 and subsequent cytochrome c release was constitutive if the nuclear localization signals and DNA binding domain of this protein. Overexpression of a Nur77 dominant negative blocked both the mitochondrial translocation of Nur77 and its induction of apoptosis (Li, H. et al., Science 289, 1159-64, 2000).

DC-Based Vaccines

One avenue of exploiting MINOR inhibition is through the potential for enhancement of DC vaccines. DC-based vaccines have been tested in a number of animal models, and have also been translated to clinical medicine in several trials. To produce DC vaccines, there are two primary methods: First, precursor cells can be isolated, grown ex vivo and differentiated in culture, then subsequently re-infused; second they can be generated in vivo through the systemic administration of GM-CSF and/or Flt-3 ligand (FLT3L). While these approaches have led to some degree of tumor immunity, they have also had limitations (Borges, L. et al., J Immunol 163, 1289-97, 1999). The identification of tumor specific and tumor-associated Ags has led to therapies such as vaccination with recombinant viruses or DCs modified to express Ags but these have also had limited effects. (Marshall, J. L. et al., J Clin Oncol 18, 3964-73, 2000; Morse, M. A. et al., Cancer Invest 21, 341-9, 2003; Ridgway, D., Cancer Invest 21, 873-86, 2003).

Therapeutic cancer vaccination depends on effective transfer of Ag to DCs and trafficking of the DCs to the secondary lymphoid organs. While many clinical trials have been initiated with ex vivo generated DCs, for the most part, no long term cures have been achieved. Interestingly, DCs are more resistant to some apoptotic pathways than other cells as a result of expression of molecules such as FLICE inhibitory protein (cFLIP) which can block caspase 8 activation and the subsequent apoptotic cascade (Leverkus, M. et al., Blood 96, 2628-31, 2000). Additionally, signaling by TRANCE, or CD154 have been shown to prevent apoptosis in DC (McLellan, A. et al., Eur J Immunol 30, 2612-9, 2000; Wong, B. R. et al., J Exp Med 186, 2075-80, 1997). In spite of these, they generally have a very short lifespan in vivo. In fact, animal models and clinical trials suggest that one major issue with ex vivo expansion and loading of DCs followed by re-injection is that relatively few DCs successfully traffic to spleen or lymph nodes (Eggert, A. A. et al., Cancer Res 59, 3340-5, 1999) and those that do are rapidly cleared by host CTL (Cayeux, S. et al., Eur J Immunol 29, 225-34, 1999). In addition, NK cells can kill DCs through TRAIL mediated apoptosis (Hayakawa, Y. et al., J Immunol 172, 123-9, 2004), and further studies using labeled DCs suggest that they are replaced every 3-4 days (Kamath, A. T., Henri, S., Battye, F., Tough, D. F. & Shortman, K., Blood 100, 1734-41, 2002), implying a limited window during which T cells can encounter DCs presenting Ag. Thus, extending the longevity of DCs would allow for their improved immunogenicity.

Enhancement of Ex Vivo DC Vaccines:

DC based therapies have been investigated for tumors for which the antigen is known, through antigen-specific activation, and also for tumors for which the antigen is not known, through whole tumor cell-based activation. The use of ex vivo DC vaccines has the potential advantage of Ag loading with multiple Ags, some of which are not identified through the use of whole tumor cell lysate as a means to pulse DCs with Ag. In fact, previous studies have shown that tumor ignorance by CD8⁺ T cells can be reversed if they are exposed to antigen-pulsed DCs. (Dalyot-Herman, N., Bathe, O. F. & Malek, T. R., J Immunol 165, 6731-7, 2000) Thus, while the potential for impact on anti-tumor immunity by DCs is clear, the trafficking and survival of these DCs have been significant limiting factors in their use, thus an enhancement to their survival provides a critical improvement to this therapy

Enhancing In Vivo DC Vaccines Generated from Transduced Hematopoietic Stem Cells

Another potential avenue for enhancing immunotherapeutic responses is by improving DC survival in a model we have recently developed involving generation of DCs in vivo from hematopoietic stem-progenitor cells (termed HSCs in this application) that have been transduced with a model tumor Ag prior to transplantation followed by differentiation into DCs in vivo via administration of systemic agents. This method provides for efficient expression of Ag by DCs in vivo. The introduction of genes encoding Ag into the HSCs combines both effective delivery of Ag and also the benefits of autologous BMT (autoBMT), which is an important treatment strategy for a number of hematologic malignancies. The success of autoBMT may be due in part to the generation of a lymphopenic environment in which it is easier to re-direct the immune system towards tumor antigens, as shown with vaccines administered post BMT, including DC based vaccines. (Asavaroengchai, W., Kotera, Y. & Mule, J. J., Proc Natl Acad Sci USA 99, 931-6, 2002).

Activation of DCs is necessary to achieve a significant percentage of tumor regressors. However, activation of DCs is a double edged sword in that while it is necessary for maximal immune stimulation, that it also leads to an induction of MINOR, thereby rapidly initiating cell death. Thus, inhibiting MINOR along with administration of activational agents it is possible that we can increase the immunogenic effect of DCs. By determining first whether there are differences in induction of MINOR by some of these candidate activators, this will allow the determination of which agents are most likely to produce an enhanced effect from the inhibition of MINOR.

Previous studies have shown that tumor ignorance by CD8⁺ T cells can be reversed if they are exposed to Ag-pulsed DCs (Dalyot-Herman, N., Bathe, O. F. & Malek, T. R., J Immunol 165, 6731-7, 2000), thus it is possible that when DCs can be kept alive long enough to activate T cells in the right setting, that their stimulatory capacity in these tolerizing settings can be greatly enhanced. Other molecules that activate DCs, primarily immunostimulatory synthetic CpG oligonucleotides have shown interesting effects in the context of stimulating tumor immunity. These CpGs act through toll like receptor (TLR)9 to activate both mature and immature DCs to upregulate co-stimulatory molecules in vitro (Sparwasser, T. et al., Eur J Immunol 28, 2045-54, 1998; Bauer, M. et al., J Immunol 166, 5000-7, 2001) and in vivo to produce γIFN (Kadowaki, N., Antonenko, S. & Liu, Y. J., J Immunol 166, 2291-5, 2001). CpG oligos also inhibit apoptosis of DCs (Park, Y., Lee, S. W. & Sung, Y. C., J Immunol 168, 5-8, 2002), which may contribute to their observed enhancement of DC vaccines (Merad, M., Sugie, T., Engleman, E. G. & Fong, L., Blood 99, 1676-82, 2002). In addition to these DC stimulators, some more recently described agents stimulate DCs through TLR7/8 (Doxsee, C. L. et al., J Immunol 171, 1156-63, 2003) and appear to produce a potent effect, possibly working through the selective stimulation of plasmacytoid DCs (Gibson, S. J. et al., Cell Immunol 218, 74-86, 2002).

I. Definitions:

“Antigen presenting cell progenitors” refers to cells that are capable of developing into a mature antigen presenting cell, e.g., a dendritic cell. Antigen-presenting cell progenitors such as dendritic cell progenitors include, for example, bone marrow stem cells, monocytes, and partially differentiated cells such as CD 14+ or CD34+ cells. Antigen presenting cell progenitors such as dendritic cell progenitors may be differentiated into mature cells by adding to the culture medium or stimulating the production of compounds, chemokines, and cytokines such as GM-CSF, in addition to IL-4, TGF.beta., M-CSF, G-CSF, IL-3, IL-1, TNF.alpha., CD40 ligand, LPS, flt3 ligand, SCF, FL, protein kinase C activators such as phorbol ester, and CD40 ligand, etc., and/or other compound(s), or combinations thereof, e.g., GM-CSF and IL-4; GM-CSF and TGF.beta.; GM-CSF, IL-4, and TGF.beta.; IL-3 and TNF; SCF and FL; IL-4 and TNF; FL and TNF; TNF and SCF; SCF, IL-1B, IL-3, IL-4, and IL-6; TGF-.beta. and TNF; TGF-.beta. and IL-4; GM-CSF, TNF and TGF-.beta. in bovine serum free media, etc., that induce differentiation, maturation, and proliferation of antigen presenting cells (see, e.g., Paul, Fundamental Immunology (3 ed. 1993); see also Young, Curr. Opin. Hematol. 6:135-144 (1999); Agilette et al., Haematologica 83:824-848 (1998); and Gluckman et al., Cytokines, Cell. and Mol. Ther. 3:187-196 (1997)). “Dendritic cells” are highly potent APCs (Banchereau & Steinman, Nature 392:245-251 (1998) and have been shown to be effective as a physiological adjuvant for eliciting prophylacetic or therapeutic antitumor immunity (see Timmerman & Levy, Ann. Rev. Med. 50:507-529 (1999)). In general, dendritic cells may be identified based on their typical shape (stellate in situ, with marked cytoplasmic processes (dendrites) visible in vitro), their ability to take up, process and present antigens with high efficiency and their ability to activate nave T cell responses. Dendritic cells may, of course, be engineered to express specific cell-surface receptors or ligands that are not commonly found on dendritic cells in vivo or ex vivo, and such modified dendritic cells are contemplated by the present invention.

Dendritic cells can be categorized as “immature” and “mature” cells, which allows a simple way to discriminate between two well-characterized phenotypes. However, this nomenclature should not be construed to exclude all possible intermediate stages of differentiation. Immature dendritic cells are characterized as APC with a high capacity for antigen uptake and processing, which correlates with the high expression of Fc.gamma. receptor and mannose receptor. The mature phenotype is typically characterized by a lower expression of these markers, but a high expression of cell surface molecules responsible for B and T cell activation such as class I and class II MHC molecules, adhesion molecules (e.g., CD54, CD 18, and CD11) and costimulatory molecules (e.g., CD40, CD80, CD83, CD86 and 4-1BB).

A “cytokine or chemokine that promotes differentiation of antigen presenting cell progenitors” refers to any cytokine or chemokine that drives stem cells or partially differentiated cells to a more mature or differentiated APC, e.g., dendritic cell, phenotype, e.g., a compound that drives a stem cell or a partially differentiated cell to an immature or mature dendritic cell phenotype. The cytokine can be provided exogenously to the cell culture, or can be provided by cells in the culture that express the cytokine or chemokine, either an endogenous or a recombinant protein. For expression of a recombinant protein, cells in the culture are transfected with an expression vector encoding the chemokine or cytokine, which then produces the protein.

“Antigen” refers to a peptide or polypeptide comprising one or more MHC class I or MHC class II epitopes. Thus, an antigen can be a protein or polypeptide, fragment of a protein or polypeptide, or a peptide comprising one or more epitopes. The antigen can be provided exogenously to the cell culture, or can be provided by cells in the culture the express the antigen, either an endogenous or recombinant protein. For expression of a recombinant protein, cells in the culture are transfected with an expression vector encoding the antigen, which then produces the protein. The antigen may be a whole protein or fragment thereof, or an MHC II epitope of about 8 to 25 amino acid residues, more preferably 9-15 amino acid residues. Dendritic cells of the invention can be pulsed with antigen either before or after administration of the adjuvant compound of formula I.

The terms “preventing” or “inhibiting” are intended to mean a reduction in cell death or a prolongation in the survival time of the cell. They also are intended to mean a diminution in the appearance or a delay in the appearance of morphological and/or biochemical changes normally associated with apoptosis. Thus, this invention provides compositions and methods to increase survival time and/or survival rate of a cell or population of cells which, absent the use of the method, would normally be expected to die. Accordingly, it also provides compositions and methods to prevent or treat diseases or pathological conditions associated with unwanted cell death in a subject.

II. Cells and Cell Culture:

The present invention provides methods of culturing and inducing maturation of antigen presenting cells (APCs) ex vivo. Specifically, the present invention is directed to methods for culturing and inducing the maturation of dendritic cells (DCs). In addition, the present invention provides methods of pulsing the cultured, matured APCs with an antigen of interest.

A. Types of Cells

Any antigen presenting cell (APC) can be used with the methods of the present invention. The term APC encompasses any cell capable of handling and presenting an antigen to lymphocytes. Typically, APCs include, e.g., Langerhans dendritic cells and Follicular dendritic cells. In addition, B cells have also been shown to have an antigen presenting function and are thus contemplated by the present invention in that should they be shown to express MINOR and that this expression led to induction of apoptosis, that inhibition of MINOR could potentially be employed to protect them from apoptosis. B cell based vaccinations are one possible means of inducing immunity. Conversely, should the presence of B cells be shown to be involved in a pathologic process, augmenting MINOR expression could be viewed as one means to eliminate B cells. In preferred embodiments of the present invention, the APCs are dendritic cells.

B. Source of Cells

APCs can be isolated from any of the tissues where they reside and which are known to those of skill in the art. In particular, dendritic cells and their progenitors may be obtained from any tissue source comprising dendritic cell precursors that are capable of proliferating and maturing in vitro into dendritic cells, when cultured and induced to mature according to the methods of the present invention. Such suitable tissue sources include, e.g., peripheral blood, bone marrow, tumor-infiltrating cells, peritumoral tissues-infiltrating cells, lymph node biopsies, thymus, spleen, skin, umbilical cord blood, monocytes harvested from peripheral blood, CD34 or CD14 positive cells harvested from peripheral blood, blood marrow or any other suitable tissue or fluid. In the context of the present invention, dendritic cells are preferably isolated from bone marrow or from peripheral blood mononuclear cells (PBMCs).

Peripheral blood can be collected using any standard apheresis procedure known in the art (see, e.g., Bishop et al., Blood 83:610:616 (1994)). PBMCs can then be prepared from whole blood samples by separating mononuclear cells from red blood cells. There are a number of methods for isolating PBMCs including, e.g., velocity sedimentation, isopyknic sedimentation, affinity purification, and flow cytometry. Typically, PBMCs are separated from red blood cells by density gradient (isopyknic) centrifugation, in which the cells sediment to an equilibrium position in the solution equivalent to their own density. For density gradient centrifugation, physiological media should be used, the density of the solution should be high, and the media should exert little osmotic pressure. Density gradient centrifugation uses solutions such as sodium ditrizoate-polysucrose, Ficoll, dextran, and Percoll (see, e.g., Freshney, Culture of Animal Cells, 3rd ed. (1994)). Such solutions are commercially available, e.g., HISTOPAQUE® (Sigma). Examples of methods for isolating dendritic cells from PBMCs are disclosed in, e.g., U.S. Pat. Nos. 6,017,527 and 5,851,756; and in O'Doherty et al., J. Exp. Med. 178:1067-1078 (1993); Young and Steinman, J. Exp. Med. 171:1315-1332 (1990); Freudenthal and Steinman, Proc. Natl. Acad. Sci. USA 57:7698-7702 (1990); Macatonia et al., Immunol. 67: 285-289 (1989); and Markowicz and Engleman, J. Clin. Invest. 85:955-961 (1990).

CD34+ PBMCs or CD14+ PBMCs can further be selected as a preferred source of dendritic cells using a variety of selection techniques known to those of skill in the art. For example, monoclonal antibodies (or any protein-specific binding protein) can be used to bind to a cell surface antigen found on the surface of the PBMC sub-population of interest (e.g., CD34 or CD14 on the surface of CD34+ or CD14+ PBMCs, respectively). Binding of such specific monoclonal antibodies allows the identification and isolation of the sub-group of PBMCs of interest from a total PBMC population by any of a number of immunoaffinity methods known to those of skill in the art. Examples of immunoaffinity methods for isolating sub-populations of PBMCs are described in, e.g., U.S. Pat. No. 6,017,527.

Alternatively, the dendritic cells of the present invention can be isolated from bone marrow. For a general description of methods for isolating dendritic cells from bone marrow see, e.g., U.S. Pat. No. 5,994,126; Dexter et al., in Long-Term Bone Marrow Culture, pages 57-96, Alan R. Liss, (1984); and Lutz et al., J. Immunol. Methods 223:77-92 (1999). Dendritic cells from bone marrow can typically be obtained from a number of different sources, including, for example, from aspirated marrow. Alternatively, bone marrow can be extracted from a sacrificed animal by dissecting out the femur, removing soft tissue from the bone and removing the bone marrow with a needle and syringe. Dendritic cells can be identified among the different cell types present in the bone marrow based on their morphological characteristics. For example, cultured immature dendritic cells in one or more phases of their development are loosely adherent to plastic, flattening out with a stellate shape.

In a preferred embodiment, the present invention provides methods to grow large numbers of murine dendritic cells from mouse bone marrow-derived dendritic cell progenitors. In another preferred embodiment, the present invention provides methods to grow large number of human dendritic cells obtained from CD14 positive human peripheral blood monocyte precursors or CD34+ progenitors.

Optionally, prior to culturing the cells, the tissue source can be pre-treated to remove cells that may compete with the proliferation and/or the survival of the dendritic cells or of their precursors. Examples of such pre-treatments are described, e.g., in U.S. Pat. No. 5,994,126.

C. Number of Days

Those of skill in the art will recognize that APCs can be cultured for any suitable amount of time. Typically, APCs are cultured from 4 to 15 days. In a preferred embodiment, the APCs of the invention are cultured for 5-7 days (Inaba et al., J. Exp. Med. 176:1693 (1992); Inaba et al., J. Exp. Med. 175:1157 (1992); Inaba et al., Current Protocols Immunol., Unit 3.7 (Coico et al., eds. 1998); Schneider et al., J. Immunol. Meth. 154:253 (1992)). In another preferred embodiment, the APCs of the invention are cultured for 10-12 days (Lutz et al., supra).

D. Compounds Added: Adjuvants and Growth Factors

1. Cytokines and Chemokines

GM-CSF has been found to promote the proliferation in vitro of both nonadherent immature dendritic cells and adherent macrophages (see, e.g., U.S. Pat. No. 5,994,126; and Lutz et al., supra). In the context of the present invention, precursor dendritic cells are thus preferably cultured in the presence of GM-CSF at a concentration sufficient to promote their survival and proliferation. The dose of GM-CSF depends, e.g., on the amount of competition from other cells (especially macrophages and granulocytes) for the GM-CSF, and on the presence of GM-CSF inactivators in the cell population (see, e.g., U.S. Pat. No. 5,994,126). The GM-CSF concentration is typically of about 1 ng/ml to 100 ng/ml, preferably of about 5 ng/ml to about 20 ng/ml. GM-CSF can be obtained from different sources well known to those of skill in the art (see, e.g., Lutz et al., supra; and U.S. Pat. No. 5,994,126).

In addition to GM-SCF, a variety of cytokines have been shown to induce the proliferation and/or maturation of dendritic cells and other APCs, and are suitable for use with the methods of the present invention (see, e.g., Caux et al., J. Exp. Med. 180:1263-1272 (1984); Allison, Archivum Immunologiae et Therapiae Experimentalis 45:141-147 (1997)). Cytokines that can be used to enhance the maturation of dendritic cells ex vivo include, but are not limited to, TNF-alpha, stem cell factor (SCF; also named c-kit ligand, steel factor (SF), mast cell growth factor (MGF); see, e.g., EP 423,980; and U.S. Pat. No. 6,017,527), granulocyte colony-stimulating factor (G-CSF), monocyte-macrophage colony-stimulating factor (M-CSF), as well as a number of interleukins, such as, e.g., IL-1.alpha. and IL-1.beta., IL-3, IL-4, IL-6, and IL-13 (see, e.g., U.S. Pat. Nos. 6,017,527 and 5,994,126). In addition to promoting the maturation of dendritic cells, some interleukins (e.g., IL-4) have been shown to suppress the overall growth of macrophages and thus favors higher levels of pure DC growth. Cytokines are used in amounts which are effective in increasing the proportion of dendritic cells present in the culture by enhancing either the proliferation or the survival of dendritic cell precursors.

In preferred embodiments, the dendritic cell precursors of the present invention are cultured in the presence of GM-CSF. In other preferred embodiments, the dendritic cells of the present invention are cultured in the presence of both GM-CSF and IL-4. When human dendritic precursor cells are cultured, the GM-CSF is preferably human GM-CSF (huGM-CSF).

2. Adjuvants

The present invention is further based, at least in part, on the discovery that a variety of adjuvants can be used to stimulate the maturation ex vivo of immature dendritic cells cultured as described above. Specifically, immature dendritic cells can be harvested from the induction cultures described supra and their maturation to end-stage antigen presenting cells can be induced by treating the cells with a variety of adjuvants. Adjuvants that promote the maturation of dendritic cells include, but are not limited to, MPL® immunostimulant and selected synthetic lipid A analogs such as aminoalkyl glucosamide phosphate (AGP). Synthetic lipid A analogs include, for example, lipid A monosaccharide synthetics such as RC-529, RC-544 and RC-527, and the disaccharide mimetic, RC-511. These adjuvants are typically used as 10% ethanol-in-water formulations, although any other formulation that promotes the maturation of dendritic cells is suitable for use with the methods of the present invention. Adjuvants that can be used with the methods of the present invention can be synthesized or obtained from a variety of sources (see, e.g., Lutz et al., supra; Johnson et al., Bioorganic Medicinal Chemistry Letters 9:2273-2278 (1999)).

E. Description of the Maturation of DCs

The maturation of DCs can be followed using a number of molecular markers and of cell surface phenotypic alterations. These changes can be analyzed, for example, using flow cytometry techniques. Typically, the maturation markers are labeled using specific antibodies and DCs expressing a marker or a set of markers of interest can be separated from the total DC population using, for example, cell sorting FACS analysis. Markers of DC maturation include genes that are expressed at higher levels in mature DCs compared to immature DCs. Such markers include, but are not limited to, cell surface MHC Class II antigens (in particular HLA-DR), ICAM-1, B7-2, costimulating molecules such as CD 40, CD 80, CD 86, CD 83, cell trafficking molecules such as CD 54, CD 11c and CD 18, etc. Furthermore, mature dendritic cell can be identified based on their ability to stimulate the proliferation of naive allogeneic T cells in a mixed leukocyte reaction (MLR).

In addition, it has been shown that, in general, while immature dendritic cells are very efficient at antigen uptake but are poor antigen presenting cells, mature dendritic cells are poor at antigen uptake but are very efficient antigen presenting cells. The antigen presenting function of a dendritic cell can be measured using antigen-dependent, MHC-restricted T cell activation assays as described herein, as well as other standard assays well known to those of skill in the art. T cell activation can further be determined, e.g., by measuring the induction of cytokine production by the stimulated dendritic cells. The stimulation of cytokine production can be quantitated using a variety of standard techniques, such as ELISA, well known to those of skill in the art.

F. General Cell Culture Methods

The present invention relies on routine techniques in the field of cell culture, and suitable conditions can be easily determined by those of skill in the art (see, e.g., Freshney et al., Culture of Animal Cells, 3rd ed. (1994)). In general, the cell culture environment includes consideration of such factors as the substrate for cell growth, cell density and cell contract, the gas phase, the medium, the temperature, and the presence of growth factors.

Exemplary cell culture conditions for dendritic cells and dendritic cell precursors are described in, e.g., U.S. Pat. Nos. 6,017,527 and 5,851,756; Inaba et al., J. Exp. Med. 176:1693 (1992); Inaba et al., J. Exp. Med. 175:1157 (1992); Inaba et al., Current Protocols Immunol., Unit 3.7 (Coico et al., eds. 1998); Schneider et al., J. Immunol. Meth. 154:253 (1992); and Lutz et al., supra.

The cells of the invention can be grown under conditions that provide for cell to cell contact. In a preferred embodiment, the cells are grown in suspension as three dimensional aggregates. Suspension cultures can be achieved by using, e.g., a flask with a magnetic stirrer or a large surface area paddle, or on a plate that has been coated to prevent the cells from adhering to the bottom of the dish. For example, the cells may be grown in Costar dishes that have been coated with a hydrogel to prevent them from adhering to the bottom of the dish.

For cells that grow in a monolayer attached to a substrate, plastic dishes, flasks, roller bottles, or microcarriers are typically used. Other artificial substrates can be used such as glass and metals. The substrate is often treated by etching, or by coating with substances such as collagen, chondronectin, fibronectin, laminin or poly-D-lysine. The type of culture vessel depends on the culture conditions, e.g., multi-well plates, petri dishes, tissue culture tubes, flasks, roller bottles, microcarriers, and the like. Cells are grown at optimal densities that are determined empirically based on the cell type.

Important constituents of the gas phase are oxygen and carbon dioxide. Typically, atmospheric oxygen tensions are used for dendritic cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media, and is typically present at a concentration of 1-10% in the incubator. The preferred CO.sub.2 concentration for dendritic cell cultures is 5%.

Cultured cells are normally grown in an incubator that provides a suitable temperature, e.g., the body temperature of the animal from which is the cells were obtained, accounting for regional variations in temperature. Generally, 37.degrees.C. is the preferred temperature for dendritic cell culture. Most incubators are humidified to approximately atmospheric conditions.

Defined cell media are available as packaged, premixed powders or presterilized solutions. Examples of commonly used media include Iscove's media, RPMI 1640, DMEM, and McCoy's Medium (see, e.g., GibcoBRL/Life Technologies Catalogue and Reference Guide; Sigma Catalogue). Defined cell culture media are often supplemented with 5-20% serum, e.g., human, horse, calf, or fetal bovine serum. The culture medium is usually buffered to maintain the cells at a pH preferably from about 7.2 to about 7.4. Other supplements to the media include, e.g., antibiotics, amino acids, sugars, and growth factors (see, e.g., Lutz et al., supra).

As described above, GM-CSF is typically added in concentrations ranging from 5 ng/ml to about 20 ng/ml. Other factors described herein and known to stimulate growth of dendritic cells may be included in the culture medium. Some factors will have different effects that are dependent upon the stage of differentiation of the cells, which can be monitored by testing for differentiation markers specific for the cell's stage in the differentiation pathway. GM-CSF is preferably present in the medium throughout culturing. Other factors that may be desirable to add to the culture medium include, but are not limited to, granulocyte colony-stimulating factor (G-CSF), M-CSF, TNF-.alpha., IFN-.gamma., IL-1, IL-3, IL-6, SCF, LPS, and thrombopoietin. In some embodiments of the present invention, IL-4 is added to the culture medium, preferably at a concentration ranging from 1-100 ng/ml, most preferably from about 5 to about 20 ng/ml.

The present invention is also based in part on the surprising result that dendritic cell can be recovered and used after cryogenic storage. The present invention, thus, also provides methods for cryogenically storing precultured DCs, e.g., in liquid nitrogen, for several weeks. In a preferred embodiment, the dendritic cells are cultured in the presence of GM-CSF, preferably for 10 days, prior to being stored cryogenically. The DCs can be stored either as immature cells or, preferably, as matured APCs, following stimulation by suitable adjuvants, as described above. Furthermore, the DCs can be cryogenically stored either before or following exposure to an antigen of interest.

A variety of cryopreservation agents can be used and are described in, e.g., U.S. Pat. No. 5,788,963. Controlling the cooling rate, adding cryoprotective agents and/or limiting the heat of fusion phase where water turns to ice help preserve the function of the activated DCs. The cooling procedure can be carried out by use of, e.g., a programmable freezing device or a methanol bath procedure. After thorough freezing, cells can be rapidly transferred to a long-term cryogenic storage vessel. The samples can be cryogenically stored, for example, in liquid nitrogen (−196.degree.C.) or its vapor (−165.degree.C.). Such storage is greatly facilitated by the availability of highly efficient liquid nitrogen refrigerators. For a general description of methods to store DCs cryogenically see, e.g., U.S. Pat. No. 5,788,963.

IV. Antigen Stimulation

A. Pulsing the Antigen Presenting Cells with an Antigen of Interest

Following expansion in culture and maturation, the APCs of the present invention can further be pulsed with an antigen. APCs pulsed with an antigen of interest will process and present epitopes of the antigen. Antigens can be from any source, including, e.g., viruses, bacteria, parasites, etc. In one embodiment, the antigen is derived from Mycobacterium sp, Chlamydia sp., Leishmania sp., Trypanosoma sp., Plasmodium sp., or a Candida sp. APCs can be pulsed with either the entire peptide (antigen) or with a fragment thereof having immunogenic properties, e.g., an epitope.

Briefly, the antigen-activated APCs (e.g., antigen-activated dendritic cells) of the invention are produced by exposing, in vitro, an antigen to the APCs (e.g., the dendritic cells) prepared according to the methods of the invention. Dendritic cells, for example, are plated in culture dishes and exposed to an antigen of interest in a sufficient amount and for a sufficient period of time to allow the antigen to bind to the dendritic cells. The amount and time necessary to achieve binding of the antigen to the dendritic cells may be determined by using standard immunoassays or binding assays. Any other method known to those of skill in the art may also be used to detect the presence of antigen on the dendritic cells following their exposure to the antigen. Methods for pulsing dendritic cells with an antigen of interest are described, e.g., in U.S. Pat. No. 6,017,527.

B. Obtaining the Antigens

In general, antigens and fragments thereof may be prepared using any of a variety of procedures well known to those of skill in the art. For example, antigens can be naturally occurring and purified from a natural source.

Alternatively, antigens and fragments thereof can be produced recombinantly using a DNA sequence that encodes the antigen, which has been inserted into an appropriate expression vector, i.e., a vector which contains the necessary elements for the transcription and translation of the inserted coding sequence, and expressed in an appropriate host. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning: A Laboratory Manual, Cold Spring Harbor Laboratories, Cold Spring Harbor, N.Y. (1989); and Ausubel et al., Current Protocols in Molecular Biology (1995 supplement).

In addition, antigens and portions thereof may also be generated by synthetic means. Synthetic polypeptides having fewer than about 100 amino acids, and generally fewer than about 50 amino acids, may be generated using techniques well known in the art. For example, such polypeptides may be synthesized using any of the commercially available solid-phase techniques, such as the Merrifield solid-phase synthesis method, where amino acids are sequentially added to a growing amino acid chain (see Merrifield, J. Am. Chem. Soc. 85:2149-2146 (1963)). Equipment for automated synthesis of polypeptides is commercially available from suppliers such as Perkin Elmer/Applied BioSystems Division, Inc., Foster City, Calif., and may be operated according to the manufacturer's instructions. Variants of a native antigen may generally be prepared using standard mutagenesis techniques, such as oligonucleotide-directed site-specific mutagenesis. Sections of the DNA sequence may also be removed using standard techniques to permit preparation of truncated polypeptides.

Within certain embodiments, the antigen of interest may be a fusion protein that comprises multiple polypeptides. A fusion protein may, for instance, include an antigen and a fusion partner which may, e.g., assist in providing T helper epitopes, and/or assist in expressing the protein at higher yields than the native recombinant protein. Other fusion partners may be selected so as to increase the solubility of the protein or to enable the protein to be targeted to desired intracellular compartments. Still further fusion partners include affinity tags, which facilitate the purification of the protein. Fusion proteins may generally be prepared using standard techniques, including chemical conjugation. Preferably, a fusion protein is expressed as a recombinant protein.

Furthermore, epitopes for use with the methods of the present invention can be selected based on the presence of specific MHC I and MHC II motifs well known to those of skill in the art.

C. Selecting the Antigens

In the context of the present invention, the antigens, antigen fragments or fusion proteins used to pulse the dendritic cells are preferably immunogenic, i.e., they are able to elicit an immune response (e.g., cellular or humoral) in a patient, such as a human, and/or in a biological sample (in vitro). In particular, antigens that are immunogenic (and portions of such antigens that are immunogenic) comprise an epitope recognized by a B-cell and/or a T-cell surface antigen receptor. Antigens that are immunogenic (and immunogenic portions of such antigens) are capable of stimulating cell proliferation, interleukin-12 production and/or interferon-.gamma. production in biological samples comprising one or more cells selected from the group of T cells, NK cells, B cells and macrophages, where the cells have been previously stimulated with the antigen.

A variety of standard assays for measuring the immunogenic properties of a polypeptide of interest or of a portion thereof are available and known to those of skill in the art (see, e.g., Paul, Fundamental Immunology, 3d ed., Raven Press, pp. 243-247 (1993), and references cited therein).

V. Immune Responses Elicited by DCs

In one aspect of the invention, the activated antigen presenting cells (e.g., the activated dendritic cells) are used to generate an immune response to an antigen of interest. An immune response to an antigen of interest can be detected by examining the presence, absence, or enhancement of specific activation of CD4+ or CD8+ T cells or by antibodies. Typically, T cells isolated from an immunized individual by routine techniques (e.g., by Ficoll/Hypaque density gradient centrifugation of peripheral blood lymphocytes) are incubated with an antigen. For example, T cells may be incubated in vitro for 2-9 days (typically 4 days) at 37.degree.C. with the antigen. It may be desirable to incubate another aliquot of a T cell sample in the absence of the antigen to serve as a control.

Specific activation of CD4+ or CD8+ T cells may be detected in a variety of ways. Methods for detecting specific T cell activation include detecting the proliferation of T cells, the production of cytokines, or the generation of cytolytic activity (i.e., generation of cytotoxic T cells specific for an antigen). For CD4+ T cells, a preferred method for detecting specific T cell activation is the detection of the proliferation of T cells. For CD8+ T cells, a preferred method for detecting specific T cell activation is the detection of the generation of cytolytic activity.

Detection of the proliferation of T cells may be accomplished by a variety of known techniques. For example, T cell proliferation can be detected by measuring the rate of DNA synthesis. T cells which have been stimulated to proliferate exhibit an increased rate of DNA synthesis. A typical way to measure the rate of DNA synthesis is, for example, by pulse-labeling cultures of T cells with tritiated thymidine, a nucleoside precursor which is incorporated into newly synthesized DNA. The amount of tritiated thymidine incorporated can be determined using a liquid scintillation spectrophotometer. Other ways to detect T cell proliferation include measuring increases in interleukin-2 (IL-2) production, Ca2+ flux, or dye uptake, such as 3-(4,5-dimethylthiazol-2-yl-)-2,5-diphenyltetrazolium. Alternatively, synthesis of lymphokines (e.g., interferon-gamma (IFN-.gamma.)) can be measured or the relative number of T cells that can respond to the antigen may be quantified.

The secretion of IL-2 or IFN-.gamma. can be measured by a variety of known techniques, including, but not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “western blot” method of Gordon et al. (U.S. Pat. No. 4,452,901); immunoprecipitation of labeled ligand (Brown et al., J. Biol. Chem. 255:4980-4983 (1980)); radioimmunoassays (RIA); enzyme-linked immunosorbent assays (ELISA) as described, for example, by Raines et al., J. Biol. Chem. 257:5154-5160 (1982); immunocytochemical techniques, including the use of fluorochromes (Brooks et al., Clin. Exp. Immunol. 39:477 (1980)); and neutralization of activity (Bowen-Pope et al., Proc. Natl. Acad. Sci. USA 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

Methods of Preventing or Inhibiting Expression of the MINOR Gene in Dendritic Cells:

A) Small Interfering RNA Technology:

Modulation of gene expression by endogenous, noncoding RNAs is increasingly appreciated as a mechanism playing a role in eukaryotic development, maintenance of chromatin structure and genomic integrity (McManus, 2002). Recently, techniques have been developed to trigger RNA interference (RNAi) against specific targets in mammalian cells by introducing exogenously produced or intracellularly expressed siRNAs (Elbashir, 2001; Brummelkamp, 2002). These methods have proven to be quick, inexpensive and effective for knockdown experiments in vitro and in vivo (2 Elbashir, 2001; Brummelkamp, 2002; McCaffrey, 2002; Xia, 2002). The ability to accomplish selective gene silencing now allows the use of siRNAs to suppress gene expression for therapeutic benefit (Xia, 2002; Jacque, 2002; Gitlin, 2002). In the context of the present invention, siRNAs have been developed which prevent or inhibit the expression of the MINOR gene in dendritic cells, potentiating the lifespans of siRNA treated dendritic cells, and thereby increasing their immunogenicity.

Use of this strategy results in markedly diminished in vitro and in vivo expression of targeted MINOR alleles. This strategy is useful in reducing expression of targeted MINOR alleles in order to model biological processes or to provide therapy for human diseases. For example, this strategy can be applied to a number of immunological disorders and/or disease, including the ability to prolong the immune response (i.e., inhibit MINOR expression) or diminish the immune response (i.e., up-regulate MINOR expression). As used herein the term “substantial silencing” means that the mRNA of the targeted MINOR allele is inhibited and/or degraded by the presence of the introduced siRNA, such that expression of the targeted allele is reduced by about 10% to 100% as compared to the level of expression seen when the siRNA is not present. Generally, when an allele is substantially silenced, it will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the siRNA is not present. As used herein the term “substantially normal activity” means the level of expression of an allele when an siRNA has not been introduced to a dendritic cell. It should be noted that others forms of anti-sense technology are included as embodiments of the present invention to inhibit MINOR gene expression in dendritic cells.

B) Identification of Proteins and Small Molecules:

The present invention provides a method (also referred to herein as a “screening assay”) for identifying modulators, e.g., candidate or test compounds or agents (e.g., peptides, peptidomimetics, small molecules, or other drugs) which have a stimulatory (increase) or inhibitory (decease) effect on the expression levels of the MINOR gene in dendritic cells.

In another embodiment, an assay is a cell-based assay in which a dendritic cell which expresses the MINOR gene is contacted with a test compound and the ability of the test compound to modulate the expression of the MINOR gene is determined. The test compounds of the present invention can be obtained using any of the numerous approaches in combinatorial library methods known in the art, including: biological libraries; spatially addressable parallel solid phase or solution phase libraries; synthetic library methods requiring deconvolution; the one-bead one-compound library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 35 12:145). Aptamer libraries may be generated as described in U.S. Pat. No. 6,423,493, U.S. Pat. No. 5,840,867, Green and Janjic, Biotechniques (2000) 39(5): 1094-6 and Geyer and Brent, Methods Enzymol (2000) 328: 171-208.

Examples of methods for the synthesis of molecular libraries can be found in, for example, DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Use of these screening methods provides a means to determine agents/compounds that may alter the expression of the MINOR gene in dendritic cells. These screening methods may be adapted to large-scale, automated procedures allowing for efficient high-volume screening of potential therapeutic agents.

VII. Pharmaceutical Compositions:

In one aspect of the invention, DCs are isolated from a patient, cultured and exposed in vitro to an antigen of interest, as described above, and after expansion and/or cryogenic storage are administered back to the patient to stimulate an immune response, including T cell activation, in vivo (see, e.g., Thurner et al., J. Immunol. Methods 223: 1-15 (1999)).

The DCs obtained as described above are exposed ex vivo to an antigen, washed and administered to elicit an immune response or to augment an existing, albeit weak, response. As such, the DCs may constitute a vaccine and/or an immunotherapeutic agent. DCs presenting an antigen of interest can be administered using a variety of routes such as, for example, via intravenous infusion. The immune response of the patient can be monitored following DC administration. Infusion can be repeated at desired intervals based upon the patient's immune response. Methods for administering dendritic cells to a patient for eliciting an immune response in the patient are described, e.g., in U.S. Pat. Nos. 5,849,589; 5,851,756; 5,994,126; and 6,017,527.

In addition, antigen presenting cells (APCs) and in particular dendritic cells can be used as delivery vehicles for administering pharmaceutical compositions and vaccines. In this context, the APCs may, but need not, be genetically modified, e.g., to increase the capacity for presenting the antigen, to improve activation and/or maintenance of the T cell response and/or to be immunologically compatible with the receiver (i.e., matched HLA haplotype). APCs may generally be isolated from any of a variety of biological fluids and organs as described above, and may be autologous, allogeneic, syngeneic or xenogeneic cells.

APCs may generally be transfected with a polynucleotide encoding a antigen of interest (or portion thereof) such that the antigen, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460 (1997). Antigen loading of dendritic cells may be achieved by incubating dendritic cells or progenitor cells with the antigen, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

### Example 1

### Analysis of Genes Upregulated in DCs

DCs have many known properties that allow them to be potent APCs. Not surprisingly, in gene analysis, molecules such as CD86, MHCII, and CD40 become upregulated upon activation. In order to elucidate other genes that are important for DC function, a subtractive hybridization analysis was undertaken, in which gene expression by activated DCs was compared to that of activated macrophages.

A) MINOR Expression in Mature DCs

Initial studies utilized a subtractive hybridization strategy to identify genes that were selectively upregulated in mature DCs relative to activated macrophages. The goal of the subtractive hybridization study was to identify new genes that were specifically upregulated in mature DCs as compared to less potent APCs (macrophages) and, thus, might contribute to the unique function of these cells.

The basic subtractive hybridization strategy for identifying genes particular to DCs has been previously published (Tseng, S. Y. et al., J Exp Med 193, 839-46, 2001). For all experiments described herein, the basic procedure for generating DCs was as follows: bone marrow cells were flushed from the femurs and tibias of mice, washed and cultured in 100-mm dishes (1×10⁶ cells/m) in 15 ml of complete medium (RPMI-1640 supplemented with 2 mM L-glutamine, 100 U/ml penicillin, 100 mg of streptomycin, 50 mM 2-ME, and 5% FCS (all from Life Technologies) supplemented with recombinant mouse GM-CSF (1000 U/ml; R&D). Nonadherent cells were removed on days 2 and 4 and replaced with fresh medium. Depending on the experiment, either additional maturation agents or viral transduction was then conducted, as specified for each individual study. The initial screen identified 114 clones specifically upregulated in DCs (data not shown), which were then further screened for redundancy and known function. Searches for homology to known genes revealed a number of interesting candidate genes for analysis.

B) Virtual Northern Analysis of Selectively Expressed Genes

After more extensive analysis of the sequences that were upregulated in DCs, the number of candidate cDNA clones to further investigate was narrowed to 36. In order to confirm selective expression of genes that were identified by the subtractive hybridization in DCs, multiple tissues from different origins were analyzed for comparative expression of the new clones and also of some with known function (e.g., CD80 and CCR7) via virtual Northern analysis of the cDNAs from the different tissues. MINOR expression was found to be quite selective for DCs (data not shown).

C) Quantitative PCR Analysis of MINOR (In Vitro)

In order to confirm the relative expression levels of MINOR between DCs and activated macrophages, qPCR analysis was developed and conducted. After differentiation into either macrophages or DCs, cells were sorted for purity, total RNA was extracted with TRIZOL, and cDNA synthesis was conducted with reagents from Roche/Applied Biosystems, and qPCR was conducted using the BioRad iCycler system with the standard detection system and reagents for SYBR green quantification. Primers were obtained from Applied Biosystems: The MINOR primer sequences are: forward: 5′AGCAGCTTAAAGGACCACCA 3′ (SEQ ID NO: 4) and reverse: 5′GGGTGTCAAGGAAGAGCTTG3′(SEQ ID NO: 5). Values have been normalized to actin.

D) Comparative Expression of Nurr77 and MINOR in DCs

Since MINOR is a member of the Nur77 family, efforts were made to determine whether Nur77 was also expressed, in order to assess redundancy of these genes. qPCR analysis was conducted as above to determine expression of Nur77 by DCs. Preparation of DCs, macrophages, and qPCR analysis was all as above. Nur77 qPCR was conducted essentially as described above, with primer sequences: TGATGTTCCCGCCTTTGC (SEQ ID NO. 6) and GCAAAGGCGGGAACATCA (SEQ ID NO. 7). For stimulation, LPS (25 ng/ml), IL-4 (500 u/ml), or TNF-α (500 u/ml) was added to day 8 cultures for 24-48 hrs prior to harvest of cells for RNA isolation.

As expected, expression of Nur77 was high in the thymus, consistent with its role in T cell apoptosis. However, in DCs, expression of Nur77 was very low to undetectable (data not shown)—even in the activated DCs, In contrast, in unstimulated mature DCs, MINOR expression was relatively high and could be induced to even higher levels by both IL-4 and TNF-α.

### Example 2

### MINOR Sequence Identity and Expression

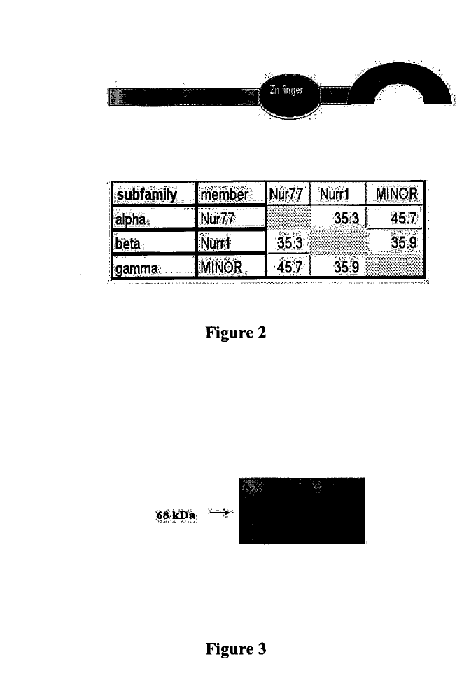

MINOR is a member of the Nur77 steroid receptor family. FIG. 2 shows a cartoon structure of MINOR, a 627-aa protein composed of an N-terminal transcriptional transactivating domain, a central zinc finger DNA binding domain with nuclear localization signals (aa290-361), and C-terminal steroid ligand binding domain (aa440-595). The figure further shows the results of protein sequence alignment of MINOR with other members of the Nur77/Nurr1 steroid hormone receptor family performed, as well as % identity to the most similar sequences.

### Example 3

### MINOR Expression at the Protein Level in Mature DCs

To verify that protein is translated for MINOR, various available antibodies for this gene family were tested. An antibody that reacts to the rat NOR-1 and mouse MINOR was identified. Utilizing this antibody, protein lysates from DCs were evaluated to assess protein expression. Mouse BMDCs were generated by standard methods and harvested at day 8. Lysates were prepared in RIPA buffer, the protein was quantified, and run on an SDS-PAGE gel. Following transfer, the blot was probed with the anti-NOR1 antibody (Santa Cruz). FIG. 3 shows an immunoblot with the predicted size band of 68 kDa present in mature DCs. MINOR is, indeed, expressed at the protein level in mature DCs.

### Example 4

### Nor-1 Expression in Mature Murine and Human DCs

BMDCs were generated from culturing bone marrow cells with GM-CSF for 6 days and then cultured for another 2 days with or without any stimuli. Briefly, BM cells were flushed from the femurs and tibias of Balb/c mice, washed, and cultured in 100-mm dishes (1×10⁶ cells/ml) in 15 ml of complete medium (RPMI-1640 supplemented with 2 mM L-glutamine, 100 U/ml penicillin, 100 mg of streptomycin, Na pyruvate, Hepes, Non-essential amino acids, 10⁻⁵ M β-ME and 5% FCS (all purchased from Life Technologies) supplemented with recombinant mouse GM-CSF (1000 U/ml; R&D Systems, Minneapolis, Minn.). Murine Granulocyte-macrophage colony-stimulating factor (GM-CSF) and IL-4 were purchased from R&D Systems (Minneapolis, Minn.). All cultures were incubated at 37° C. in 5% humidified CO₂. Nonadherent cells were removed on day 2 and 4 and replaced with fresh medium. After 6 days of cultures, the cells in suspension were reseeded in complete medium with recombinant mouse IL-4 (1000 U/ml; R&D) for two more days. To induce maturation of the cells, GM-CSF and IL-4 or TNF-a (50 ng/mL) were added for a further 48-hour period.

Mature (MHC classII highCD86high) BMDCs were FACS-sorted and examined for their expression of Nur77, Nor-1 and Nurr-1, compared to those of immature BMDCs (MHC class II low CD86 low). After differentiation into either macrophages or DCs, cells were sorted for purity, total RNA extracted with TRIZOL, and cDNA synthesis conducted with an Invitrogen 1^{st} strand synthesis kit. 1:9 cDNA dilutions were analyzed in duplicate. Standard curves were generated on the cycle threshold (Ct) vs log ng input RNA; then all samples were calculated with n=(Ct−b)/m.

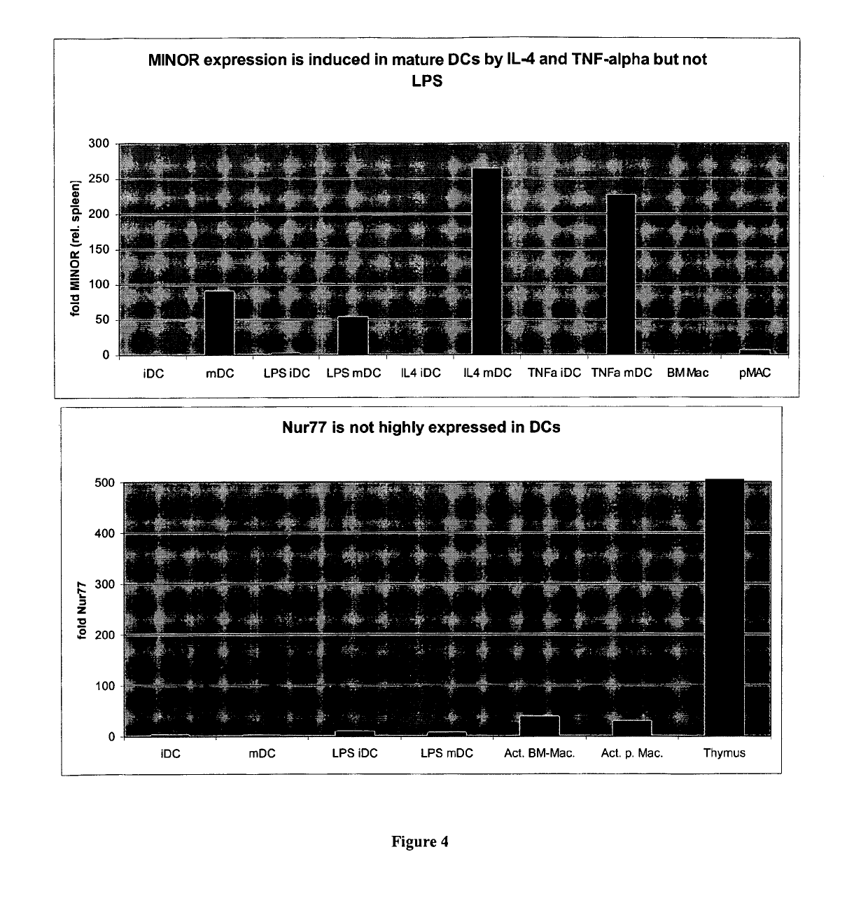

For calculating fold expression of MINOR, inverse logs were calculated and experimental values divided by 18s internal controls. Applied Biosystems PDAR reagents were used for detection of 18s rRNA and MINOR with universal Taqman Mastermix, with primers at 600 nM and probes at 200 nm. PCR was done using specific primer sets for each clone. 6FAM/TAMRA labeled probe CCCTTGCAGCCCTCGCAGGTG (SEQ ID NO: 8) was used with flanking oligos TGCCAGCACTACGGAGTCC (SEQ ID NO: 9) and TTCTGCACCGTTCTCTTGAAGA (SEQ ID NO 10) for specific detection of MINOR. Quantitative RT-PCR analysis shows mature DCs cultured in GM-CSF or GM-CSF/IL4 expressed significantly higher levels (100 fold) of Nor-1 compared to immature BMDCs (FIG. 4). Treatment of BMDCs with LPS or CpG did not promote Nor1 expression, suggesting the protective effects of LPS and CpG.

qPCR analysis was conducted as above to determine expression of Nur77 by DCs. Preparation of DCs, macrophages, and qPCR analysis was all as above. For stimulation, LPS (25 ng/ml), IL-4 (500 u/ml), or TNF-α (500 u/ml) was added to day 8 cultures for 24-48 hrs prior to harvest of cells for RNA isolation.

Expression of MINOR, but not Nurr77, was detected in various DC populations and increased in mature DC populations. Of note, neither Nurr77 nor Nur-1 was detected in any tested DC samples.

The previous results show that Nur77 is not expressed in DCs, prompting the hypothesis that the mouse MINOR substitutes for the signal to induce apoptosis, in a selective fashion.

### Example 5

### NOR-1-Induced Apoptosis in a DC-Like Cell Line

In order to determine whether forced expression of the MINOR gene would induce apoptosis in a DC-like cell line, the DC 2.4 cell line, which is phenotypically similar to immature DCs (Shen, Z., Reznikoff, G., Dranoff, G. & Rock, K. L., J Immunol 158, 2723-30, 1997) and does not express high levels of MINOR naturally, was gene-modified (transfected) with either a control GFP (pEGFP-N2) or the rat homolog MINOR cDNA, the Nor-1 GFP vector pCI Nor-1, which was obtained from Astar Winoto and inserted into HindIII-BamHI digested peGFPN2 (Clonetech). Expression of Nor-1 in these cells was achieved by plasmid DNA transfection using Lipofectamine 2000 reagent as directed (GIBCO-BRL). 2-4 days later, cells were harvested and stained with Annexin V/7-AAD using the manufacturer's recommended protocol, and analyzed by FACS for cell death. Such analysis included for the early (Annexin V+/AAD−)—lower right quadrant on the FACS plot below—and late (Annexin V+/AAD+)—upper right quadrant on plot—stages of cell death. Cells were stained with PE-conjugated annexin V and 7-AAD, according to the manufacturer's instructions, and staining was assessed by flow cytometry. Cells that were apoptotic are annexin V-positive/7-AAD-negative.

Antibodies and annexin V-PE and 7-amino-actinomycin D (7-AAD) were purchased from BD PharMingen unless specified. For the surface phenotype analyses described herein, the following fluorochrome-labeled mAb were employed: anti-CD11c (HL3), anti-Gr1 (RB6-8C5), anti-B220/CD45R (RA3-6B2), anti-CD80 (1G10), anti-CD86 (GL1), anti-1-E^{k/d} (14-4-42) mAbs (BD PharMingen, San Diego, Calif.). Prior to staining with labeled mAbs, cells were preincubated for 5 min with anti-CD 16/32 (2.4G2) mAbs to block FcγII/III receptors. Flow cytometry was performed on a FACSCalibur instrument (BD PharMingen) using CELLQUEST acquisition and data were analyzed using the software FlowJo (Tree Star, San Carlos, Calif.).

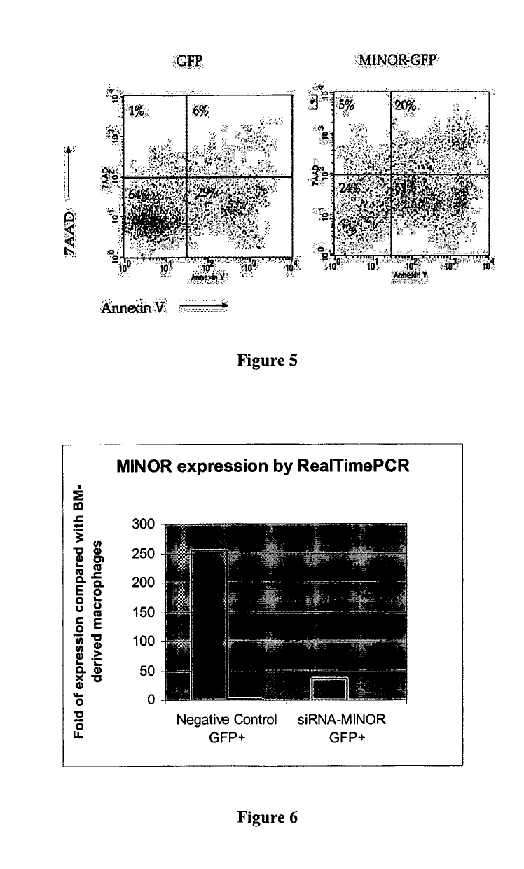

As FIG. 5 shows, there was a significant increase in both Annexin V and 7-AAD positive cells, if they constitutively expressed the Nor1 homolog of MINOR, indicating that constitutive MINOR expression induces cell death. In other words, the MINOR homolog Nor1 was shown to induce apoptosis in DC2.4 cells. It should be noted that, in all studies of apoptosis, great care was taken in controls and compensation to minimize artifact due to autofluorescence of dying cells.

### Example 6

### Generation of a Lentivirus-Based Vector Expressing siRNA to Genetically Suppress MINOR Expression

A) siRNA Design

Six different siRNAs were designed corresponding to the Nor-1 gene (GenBank accession no. NM_{—}015743). Sequences were chosen using the RNAi design software (Oligoengine, Seattle, Wash.). siRNAs with no sequence homology to any known mouse gene were used as negative controls. All siRNA sequences were BLAST searched in the National Centre for Biotechnology Information's (NCBI) “search for short nearly exact matches” mode against all mouse sequences deposited in the GenBank and were not found to have significant homology to genes other than Nor-1.

B) Generation of Lentivirus-Based siRNA

To generate a vector-based suppression of MINOR expression, the construct pSUPER-retro (Oligoengine) was employed as a template. The siRNA oligonucleotides designed contained a sense strand of 19 nucleotide sequence followed by a short spacer (TTCAAGAGA) (SEQ ID NO. 7), the reverse complement of the sense strand, and five thymidines as a RNA pol III transcriptional stop signal. Briefly, the pSUPER-retro vector was digested with BglII and HindIII and the annealed oligos (5′-GATCCCCTGCCCTTGTCCGAGCTTTATTCAAGAGATAAAGCTCGGACAAGGGCATTTTTGGAAA-3′; forward (SEQ ID NO. 2) and 5′-AGCTTTTCCAAAAATGCCCTTGTCCGAGCTTTATCTCTTGAATAAAGCTCGGACAAGGGCAGGG-3′; reverse (SEQ ID NO. 3)) were ligated into the vector according to the manufacturer's protocol. To construct lentivectors encoding the siRNA construct, the complete human H1-RNA promoter and the siRNA cassette and the PGK promoter were subcloned at XhoI and NheI 5′ of the reporter eGFP gene of the third generation self-inactivating lentiviral vector, Sin-18 provided by D. Trono (Zufferey, R. et al., J Virol 72, 9873-80, 1998). All inserts were sequenced.

A 3-plasmid transfection system was employed to generate high-titer lentivirus as previously described (Cui et al., 2003). Briefly, 293 T cells were grown to 80% confluency on a 100-mm cell culture dish and transfected with 10 μg of pCMV-8.9, 2.5 g of pMD.G, 5 μg of LV-siRNA using the Lipofectamine 2000 (Life Technologies). Supernatants containing the virus were collected at 24 and 48 hour post-transfection, pooled and filtered with 0.2-μm filter. The titer of the virus, measured in transducing units (TU), was determined using 293 T cells and analyzed by FACS analysis (by GFP).

C) qPCR Analysis of siRNA-MINOR-Transduced DCs

In order to validate that the siRNA was functionally decreasing the expression of MINOR, a quantitative PCR analysis was conducted with MINOR-specific primers. DCs were generated from bone marrow progenitor cells, as described above, transduced with the LV siRNA-MINOR-GFP on days 3-6 of culture, harvested on day 8, and evaluated by FACS for phenotype and RNA for quantitative PCR, utilizing the BioRad I cycler system. The MINOR primer sequences are: forward: 5′AGCAGCTTAAAGGACCACCA 3′ (SEQ ID NO. 11) and reverse: 5′GGGTGTCAAGGAAGAGCTTG3′ (SEQ ID NO. 12). Values were normalized to actin. FIG. 6 shows that, while unmodified mature DCs express a high level of MINOR, transduction with siRNA-MINOR led to a 90% knockdown in expression.

### Example 7

### Transduction of DC2.4 with Lentivirus Expressing MINOR-Targeted siRNA

In order to provide further evidence that the siMINOR specifically down-regulated MINOR expression, the ability of the siRNA-MINOR to downregulate the expression of MINOR in a controlled setting was tested. Since DC 2.4 cells normally express no detectable level of MINOR but undergo apoptosis when transduced with the rat homolog for MINOR, NOR-1, the effects of co-transducing DC2.4 cells with MINOR and the siRNA-MINOR or siRNA-control were investigated.

The DC2.4 cells were transduced with siRNA-MINOR or LV-control-siRNA (MOI=5; 2 times), followed by transient transfection of rat Nor-1 into the cells using lipofectamine 2000. Transfected cells were stained for apoptotic markers (annexin V and 7-AAD) 48 hr post-transfection. Transduced DC2.4 cells were gated based on GFP fluorescence and compared with the non-transduced DC2.4 (GFP negative). The expression of MINOR induced apoptosis via forced expression of MINOR, which was blocked in the GFP+ fraction of the siRNA-MINOR transduced cells, but not in the GFP (−) fraction, or in either the GFP+ or (−) fraction of those transduced with MINOR and the control siRNA. NOR-1 expression was measured by quantitative RT-PCR.

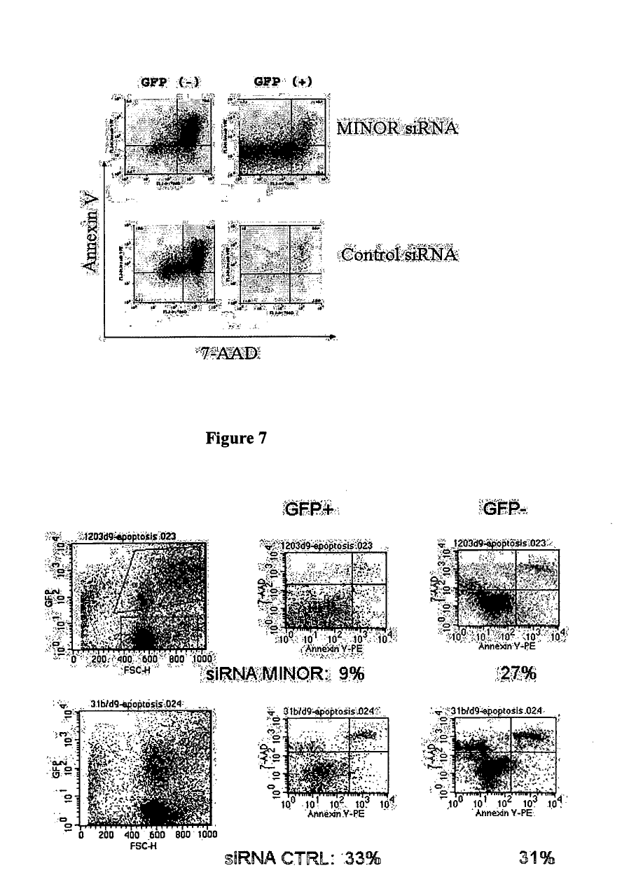

As shown in FIG. 7, only 10% of the DC2.4 transduced with LV-Nor1-siRNA were dead (annexin V+, 7AAD+) whereas 66% of the cells transduced with LV-control-siRNA were annexin V+, 7AAD+. In all cases, comparable levels of apoptosis were detected among non-transduced cells. These results indicate that transducing DC2.4 with lentivirus expressing MINOR-targeted siRNA prevents MINOR-induced DC apoptosis.

### Example 8

### Inhibition of Apoptosis in BM-Derived DCs by siRNA-MINOR

To determine whether the siRNA-MINOR would inhibit cell death in primary BM-derived cultures, BM-DCs were generated, transduced, and followed for natural apoptosis. Bone marrow DCs were generated in vitro, (as described previously). Cells were subjected to three rounds of lentiviral transduction with concentrated virus (i.e. MOI=5) on day 2, 4 and 6 in the presence of 8 μg/ml polybrene.

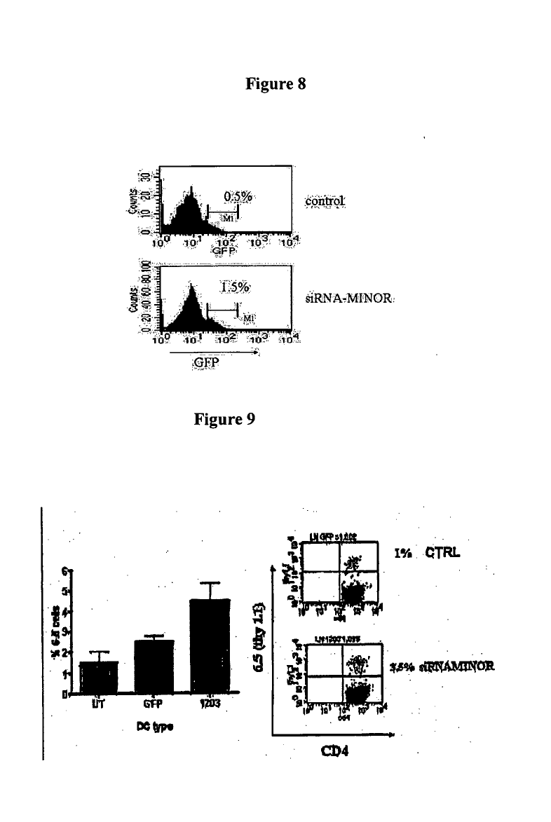

After 6 days of cultures, the cells in suspension were reseeded in complete medium with and on days 5 & 6 were transduced with the LV encoding the siRNA-MINOR-GFP or control siRNA-GFP (sequence GTATACGTGTTTGCTCCCTT (SEQ ID NO. 13), no known homology to any gene). On day 9, the cells were analyzed for induction of cell death. As the plots depicted in FIG. 8 show, there is no significant difference in cell death in the GFP⁻ fractions in both populations or the GFP+ in the control. In contrast, there is a significant decrease in cell death in the transduced (GFP⁺) fraction in the siRNA-MINOR-expressing population, indicating an inhibition of apoptosis. Accordingly, it is indicated that transduction with the siRNA can prolong DC survival.

The previous results showing that MINOR's forced expression induces apoptosis, and that its inhibition inhibits natural apoptosis in DCs, indicate that MINOR merits further investigation as a potentially significant gene both for basic DC function, in that it may help regulate DC lifespan, thereby limiting uncontrolled T cell activation, and also as a target for improving DC-based therapies.

### Example 9

### Transduction of Ex Vivo-Generated DCs with siRNA-MINOR and their Survival after Infusion

To test the hypothesis that the observed decrease in apoptosis correlates with an improved survival in vivo, BALB/c BMDCs were grown ex vivo, transduced with either the GFP control or the siMINOR-GFP on days 4 and 5, were sorted for GFP⁺/CD11c⁺, and then 5×10⁶ were infused on day 7. Four days after infusion, the mice (3/grp) were sacrificed, and the lymph nodes were harvested and analyzed for retention of labeled infused DCs to determine if there was a difference between the control and siMINOR-transduced DCs in their maintenance after infusion. Lymph nodes were enriched for DCs and then stained for CD11c. FIG. 9 shows an approximately 3-fold increase in the survival of infused DCs that expressed the siRNA for MINOR. This indicates that transduction of ex vivo-generated DCs with siRNA-MINOR improves their survival after infusion.

The use of ex vivo DC vaccines has the potential advantage of Ag loading with multiple Ags, some of which are not identified, through the use of whole tumor cell lysate as a means to pulse DCs with Ag. In fact, previous studies have shown that tumor ignorance by CD8⁺ T cells can be reversed if they are exposed to antigen-pulsed DCs. (Dalyot-Herman, N., Bathe, O. F. & Malek, T. R., J Immunol 165, 6731-7, 2000). Thus, while the potential for impact on anti-tumor immunity by DCs is clear, the trafficking and survival of these DCs have been significant limiting factors in their use. Accordingly, an enhancement to their survival could provide a critical improvement to this therapy. Improving the survival of the DC vaccines while they are being expanded in vitro and, also, after they have been infused, is further contemplated herein.

### Example 10

### Transduction of Ex Vivo DCs with siRNA-MINOR and their Vaccine Potency

In order to follow the immune responses generated by the DCs, the well-defined Influenza hemagglutinin antigen (HA) may be used, along with well-characterized anti-HA TCR transgenic (Tg) mice, to explore basic immunological processes relevant to tumor immunity. The HA Ag and the pair of MHC class I (K^{d})-(termed clone 4) and II (1-E^{d})-restricted (termed 6.5), HA-specific TCR Tg mice are utilized to track immune responses.

Of significance to the tumor studies proposed, HA behaves like a natural tumor Ag in a number of tumor models such as the A20 B cell lymphoma, in that moderate levels of HA expression do not alter the biology, immunogenicity, or in vivo growth characteristics of the tumor (Sotomayor, E. M., Borrello, I., Tubb, E., Allison, J. P. & Levitsky, H. I., Proc Natl Acad Sci USA 96, 11476-81, 1999; Staveley-O'Carroll, K. et al., Proc Natl Acad Sci USA 95, 1178-83, 1998; Adler, A. J. et al., J Exp Med 187, 1555-64, 1998). In addition, transgenic mice available that express HA as self allow investigation of the activation of tolerant T cells (Adler, A. J. et al., J Exp Med 187, 1555-64, 1998). Thus, while this Ag may have limitations, it provides a strong foundation on which to develop this system and addresses some important fundamental questions of immune responses.

Since it appeared that transduction with siRNA-MINOR could inhibit cell death, it was hypothesized that it would also enhance the immunogenicity of BM-DCs when used as a vaccine. To assess the immunogenicity of ex vivo-generated, HA-pulsed DCs, DCs were generated from BM, transduced with either GFP or siRNA-MINOR GFP, and pulsed with the class II restricted peptide for HA. To track the T cell responses, 6.5 T cells were adoptively transferred (here on a thy1.1 background), and, 5 days later, the mice were sacrificed to determine T cell expansion in spleen and lymph nodes.

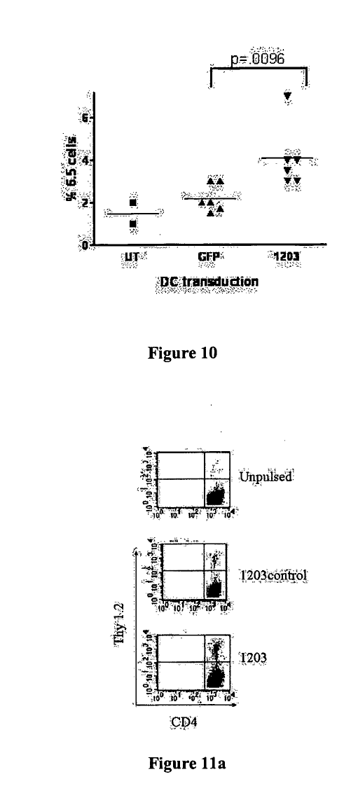

FIG. 10 shows a representative FACS plot, stained for CD4 and thy1.1 for the siRNA-MINOR (left) and control siRNA-GFP (right), as well as a bar graph showing the average and SD for all mice (2 expts, 6 mice/vector). The lower graph shows that there was a significant enhancement in stimulation of Ag-specific T cells, if the DCs expressed siRNA-MINOR along with Ag (HA in this case). Thus, siRNA-MINOR transduction of ex vivo-generated, HA-pulsed DCs enhances their immunogenicity.

In these first studies, DCs were not sorted prior to infusion; thus, only a fraction (20-30%) of the infused DCs actually expressed the gene. Additional experiments contemplated herein will determine the effect for sorted DCs. Furthermore, in order to expand the system to investigate therapeutic avenues, an A20 lymphoma has been developed that expresses the Epstein Barr Virus Ag, LMP2. Thus, a second, naturally occurring, Ag in a tumor is contemplated for future studies.

### Example 11

### Transduction of Ex Vivo-Generated DCs with siRNA-MINOR and their Capacity to Stimulate Tolerant T Cells

It was next tested whether the observed enhancement in T cell stimulation gained from transduction of ex vivo-generated DCs with si-MINOR would also result in a stronger stimulation, perhaps sufficient to activate tolerant T cells. For these studies, a similar experimental design was conducted as above, with the major difference being that the T cells used for activation were from mice that express HA as self antigen, and followed by a thy 1.1/1.2 disparity rather than by a transgenic T cell receptor antibody. Thus the stimulation of the endogenous, tolerant repertoire is measured, rather than stimulation of the transgenic T cell receptor.

2.5×10⁷ T cells from C3-HA (thy 1.2) mice were adoptively transferred into the parent strain, B10.D2 mice that carry the thy1.1 marker. B10.D2 (thy1.1) DCs were grown ex vivo from bone marrow, as before, and transduced with either the siRNA-MINOR or the siRNA-control, and then, prior to subcutaneous injection, pulsed with HA class II peptide and washed. 5 days later, mice were sacrificed and lymph nodes removed for analysis. Cells were stained with antibodies for thy 1.2 and CD4. To determine whether, indeed, these are antigen-specific responses without using transgenic T cells, the control of unpulsed DCs was added.

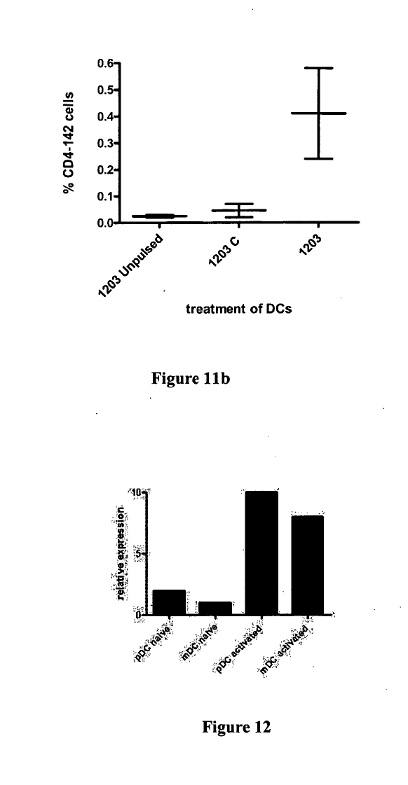

Results of these studies (FIG. 11) show that, while ex vivo unpulsed siRNA-MINOR-transduced DCs, or pulsed DCs that are control-transduced, are not sufficient to overcome the unresponsiveness in these mice, ex vivo pulsed DCs expressing siRNA-MINOR do have sufficient stimulatory capacity to overcome this unresponsiveness. The numbers of T cells of expansion in the control group is that of background detection, as shown compared to unpulsed stimulation levels, indicating that siRNA-MINOR transduction helps to impart a stronger stimulus to the DCs. In these studies, the C3-HA mice (termed 142) that express HA as a self-antigen were used as the source of adoptively transferred T cells. The peripheral T cells from the C3-HA mice are tolerant to HA by unresponsiveness, rather than deletion (Adler, A. J. et al., J Exp Med 187, 1555-64, 1998). Thus, transduction of ex vivo-generated DCs with siRNA-MINOR was found to enhance their capacity to stimulate tolerant T cells.

### Example 12

### Activation of DCs Leads to Upregulation of MINOR In Vivo

In light of the upregulation of MINOR after stimulation shown in vitro, it was next tested whether there was likewise an upregulation in vivo after systemic DC activation. BALB.c mice were treated with the TLR 7/8 agonist 3M-019 (obtained from 3M Pharmaceuticals and injected at 200 μg/mouse/day on days 0 and 2). Four days later, lymph nodes were harvested from both naïve mice and the stimulated mice and sorted into the following fractions: for the naïve mice, the LN were sorted into CD11c^{low}/CD86^{low} or CD11c^{high}/86^{low} fractions in order to isolate the less mature naïve DCs, which were then re-checked by FACS for purity, followed by RT-qPCR analysis. Similarly, the LN from the TLR-activated mice were sorted into CD11c^{low}/CD86^{high} or CD11c^{high}/86^{high} fractions and subjected to the above process, as well.

Thus, the fractions were sorted based on the intensity of their CD11c (which correlates with the plasmacytoid (low) and myeloid (CD11c high) phenotypes). FIG. 12 shows that the activation of DCs leads to upregulation of MINOR in vivo. In fact, both fractions from the TLR-activated mice contain the highest levels of MINOR. However, additional studies will more accurately separate the phenotypes to determine differences in MINOR expression.

### Example 13

### BMT with siRNA-MINOR-Transduced HSCs Leads to Stable Knockdown of MINOR in DCs In Vivo after Reconstitution

Previously presented results using the BMT system depend on being able to correlate GFP expression with decreased MINOR expression. It was, thus, sought to confirm that in the DC populations that were GFP+ after transplant with siRNA-MINOR, there was, in fact, a decrease in MINOR expression at the timepoints selected for analysis. A potential avenue for enhancing immunotherapeutic responses is by improving DC survival in a model recently developed involving generation of DCs in vivo from hematopoietic stem-progenitor cells (termed HSCs in this application) that have been transduced with a model tumor Ag prior to transplantation followed by differentiation into DCs in vivo via administration of systemic agents. This method provides for efficient expression of Ag by DCs in vivo.

The introduction of genes encoding Ag into the HSCs combines both effective delivery of Ag and also the benefits of autologous BMT (autoBMT), which is an important treatment strategy for a number of hematologic malignancies. The success of autoBMT may be due in part to the generation of a lymphopenic environment in which it is easier to re-direct the immune system towards tumor antigens, as shown with vaccines administered post BMT, including DC based vaccines (Asavaroengchai, W., Kotera, Y. & Mule, J. J., Proc Natl Acad Sci USA 99, 931-6, 2002). For all experiments describing the in vivo generation of DCs from transduced HSCs used for BMT, the following experimental procedure was conducted: BALB/c BM was harvested and enriched for HSCs using the StemSep separation kit (Stem Cell Technologies). HSCs were transduced for 3 days with either the control siRNA-GFP LV or the siRNA-MINOR GFP and then transplanted into myeloablatively (850 cGy radiated) treated BALB/c mice. After a minimum of 8-10 weeks of engraftment, the mice were sacrificed and their spleens and lymph nodes harvested for DC isolation.

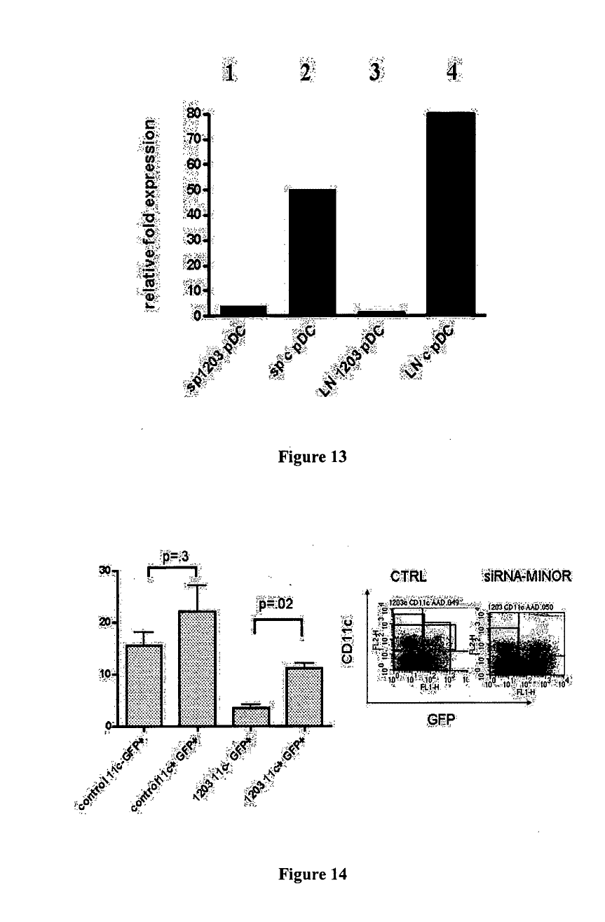

Mice (6/Group) were transplanted with HSCs transduced with either siMINOR (1203) or the control GFP vector, allowed to engraft for 10 weeks, then analyzed for knockdown. Spleen and lymph nodes were harvested from the mice, stained with CD11c and B220, then FACS sorted into GFP+ cells (thus only transduced in each group are compared here) in the plasmacytoid phenotype (CD11c^{lo}/B220⁺) or myeloid (CD11^{hi}/B220⁻). Purity of the sort was confirmed by FACS, then RNA was isolated, cDNA transcribed, and qPCR conducted. Values were normalized to actin, and the lowest value was arbitrarily set to 1. FIG. 13 shows a significant decrease in expression of MINOR RNA in the GFP⁺ population of those expressing the siRNA-MINOR vector, compared to the control.

### Example 14

### Expression of siRNA-MINOR in CD11c+ Cells

In order to assess relative expression of the siRNA-MINOR in CD11c+ cells, compared to control siRNA expression, FACS analysis was used to compare GFP by CD11c. DC subpopulations were prepared from spleens and lymph nodes of BALB/c mice using methods similar to those described previously (Sparwasser, T. et al., Eur J Immunol 28, 2045-54, 1998; Bauer, M. et al., J Immunol 166, 5000-7, 2001). Briefly, spleens were cut into small fragments, subjected to digestion by collagenase and DNase I (Roche Applied Science, Indianapolis, Ind.) at room temperature for 25 min, then treated with EDTA for 5 min. Light density cells were isolated by centrifugation in Nycodenz medium (Accurate Chemical and Scientific Corporation, Westbury, N.Y.), followed by depletion of CD3⁺ and CD19⁺ cells, then immunomagnetic bead enrichment of CD11c⁺ cells (CD11c⁺ isolation kit, Miltenyi Biotec, Auburn, Calif.). Collagenase digestion was omitted to isolate DCs from lymph nodes. The cells were then stained for CD11c and 7-AAD.

FIG. 14 shows, on the right, representative FACS plots of LN for GFP by CD11c, in order to compare relative expression of the vectors in DC (upper 2 quads) vs. non-DC (lower 2 quads) populations. The statistics show that, while there is no significant difference in the GFP expression by CD11c+ and CD11c− cells in the control group (labeled 1203c on the FACS), there is a significant difference in the siRNA-MINOR group (labeled 1203).

If no selective expression were present, then the ratio of GFP in CD 11c+ to CD11c− should be equal. However, a comparison of the percentage of CD11c+ cells that contained the vector with the percentage of CD11c− cells that contained vector showed that the there was preferential vector expression of siRNA-MINOR in CD 11c+/CD11c− compared to control GFP+ in CD11c+/CD11c−. In other words, there was a selective expression of siRNA-MINOR in CD11c+ cells, consistent with the notion that siRNA-MINOR confers a survival advantage to DCs, thereby preserving the CD11c population. Thus, transduction of HSC with MINOR siRNA confers selective protection to DC populations.

### Example 15

### siRNA-MINOR Expression in CD86^{hi} Cells

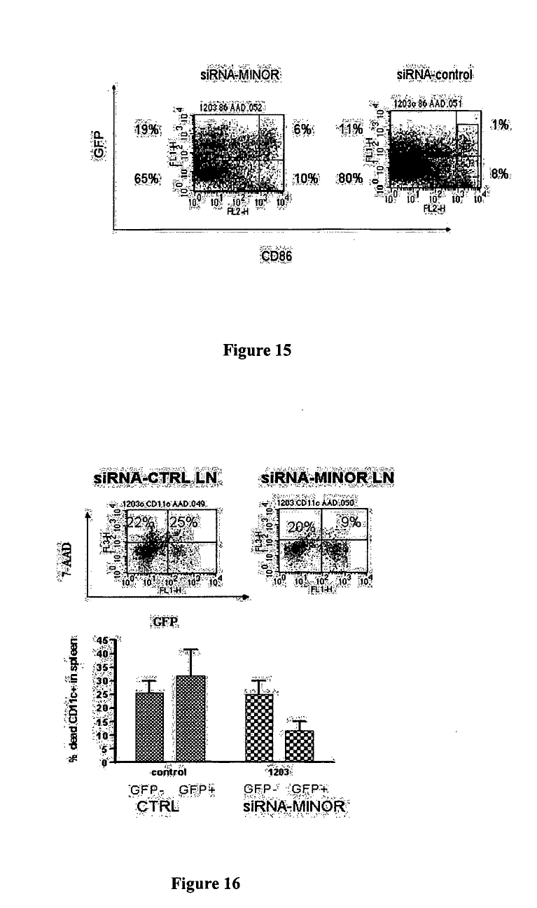

In order to assess levels of expression as related to DC maturity, a similar analysis was conducted for expression of the vectors based on CD86 expression. Again, LN cells were enriched for DCs, and then stained with CD86 and 7-AAD. The ratios of GFP+ cells in the CD86^{hi} and CD86^{lo-intmdt} were compared. The ratios of upper left to lower left shown in FIG. 15 should be the same as upper right to lower right, if no selective expression were involved. As the comparisons show, however, while in the GFP control, there is no significant difference in these ratios, in the siRNA-MINOR, there is a much higher percentage of GFP+ cells in the CD86^{hi} group, indicating that this group is the most selectively affected. This indicates that siRNA-MINOR is preferentially maintained in CD86^{hi} populations.

### Example 16

### siRNA-MINOR Increases the Viability of DC Progeny of Transduced HSCs

Following reconstitution, mice were analyzed for the percentage of CD11c⁺ cells that were alive. Two separate comparisons were made: One within each group of mice (control GFP⁺ vs control GFP⁻ and siRNA-MINOR GFP⁺ vs siRNA-MINOR GFP⁺ As FIG. 16 shows, there is a significant difference between the MINOR-siRNA GFP⁺ and GFP⁻ and the GFP⁺ vs control GFP⁺, indicating a selective protection from the siRNA-MINOR. Thus, siRNA transduction of HSCs prior to transplant results in a decrease in DC death.

### Example 17

### Human DC MINOR Expression Patterns

To address the relevance of these findings to the clinical setting, namely, inhibiting MINOR in ex vivo DC vaccines, human DCs were generated to determine whether the same pattern of specific expression of MINOR was present in human cells. In effect, the studies described for mouse were repeated with human cells. In order to maximize sensitivity in the qPCR analysis, new primers were designed for human

MINOR with the following sequence: forward: 5′ GTA TCC AGA AGCTGG GCA GA (SEQ ID NO. 13) and reverse: 5′ CTG AAG TCG ATG CAG GAC AA (SEQ ID NO. 14). Expression (normalized to actin) was compared in the following cell types: CD34⁺ hematopoietic cell progenitors, LPS activated-monocyte derived-macrophages, and activated dendritic cells.

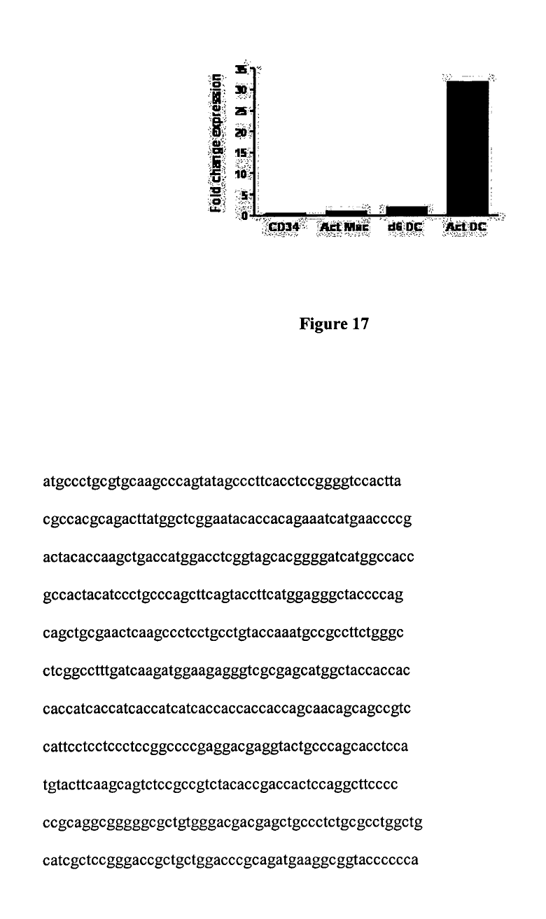

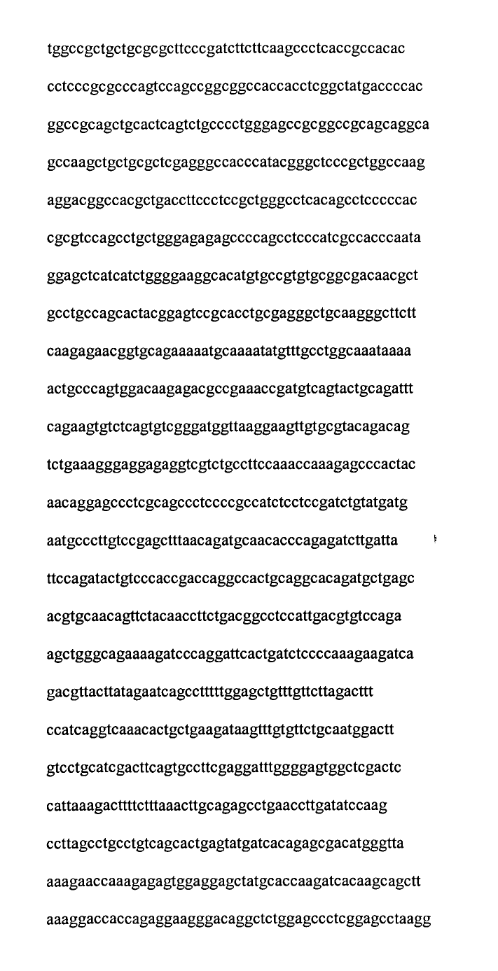

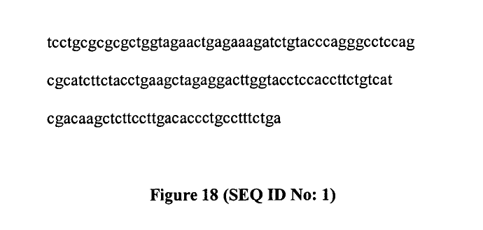

The CD34⁺ cells were obtained at 90% purity; the monocytes and dendritic cells were generated from PBMCs, grown in the presence of M-CSF (for monocytes) or GM-CSF (30 ng/ml)+IL-4 (30 U/ml) (for DCs). At day 7, 100 ng/ml of LPS was added to the cultures for activation, and cells were harvested on day 8 for FACS analysis (to assure CD14−/CD11c+ phenotype) and RNA isolation. FACS analysis confirmed >90% purity of the two cell populations. CD34 cells and macrophages were tested for comparison. RNA was transcribed into cDNA, which was used for quantitative PCR analysis. FIG. 17 shows that there is a dramatic upregulation of MINOR in activated DCs, as previously found in the murine system.

### REFERENCES

1. Tseng, S. Y. et al. B7-DC, a new dendritic cell molecule with potent costimulatory properties for T cells. J Exp Med 193, 839-46 (2001).
2. Zufferey, R. et al. Self-inactivating lentivirus vector for safe and efficient in vivo gene delivery. J Virol 72, 9873-80 (1998).
3. Borges, L. et al. Synergistic action of fms-like tyrosine kinase 3 ligand and CD40 ligand in the induction of dendritic cells and generation of antitumor immunity in vivo. J Immunol 163, 1289-97 (1999).
4. Marshall, J. L. et al. Phase I study in advanced cancer patients of a diversified prime-and-boost vaccination protocol using recombinant vaccinia virus and recombinant nonreplicating avipox virus to elicit anti-carcinoembryonic antigen immune responses. J Clin Oncol 18, 3964-73 (2000).
5. Morse, M. A. et al. Immunotherapy with autologous, human dendritic cells transfected with carcinoembryonic antigen mRNA. Cancer Invest 21, 341-9 (2003).
6. Ridgway, D. The first 1000 dendritic cell vaccines. Cancer Invest 21, 873-86 (2003).
7. Leverkus, M. et al. Maturation of dendritic cells leads to up-regulation of cellular FLICE-inhibitory protein and concomitant down-regulation of death ligand-mediated apoptosis. Blood 96, 2628-31 (2000).
8. McLellan, A. et al. MHC class II and CD40 play opposing roles in dendritic cell survival. Eur J Immunol 30, 2612-9 (2000).
9. Wong, B. R. et al. TRANCE (tumor necrosis factor [TNF]-related activation-induced cytokine), a new TNF family member predominantly expressed in T cells, is a dendritic cell-specific survival factor. J Exp Med 186, 2075-80 (1997).
10. Eggert, A. A. et al. Biodistribution and vaccine efficiency of murine dendritic cells are dependent on the route of administration. Cancer Res 59, 3340-5 (1999).
11. Cayeux, S. et al. Direct and indirect T cell priming by dendritic cell vaccines. Eur J Immunol 29, 225-34 (1999).
12. Hayakawa, Y. et al. NK cell TRAIL eliminates immature dendritic cells in vivo and limits dendritic cell vaccination efficacy. J Immunol 172, 123-9 (2004).
13. Kamath, A. T., Henri, S., Battye, F., Tough, D. F. & Shortman, K. Developmental kinetics and lifespan of dendritic cells in mouse lymphoid organs. Blood 100, 1734-41 (2002).
14. Camporeale, A. et al. Critical impact of the kinetics of dendritic cells activation on the in vivo induction of tumor-specific T lymphocytes. Cancer Res 63, 3688-94 (2003).
15. Gorski, K. et al. A set of genes selectively expressed in murine dendritic cells: utility of related cis-acting sequences for lentiviral gene transfer. Mol Immunol In press (2003).
16. Cheng, L. E., Chan, F. K., Cado, D. & Winoto, A. Functional redundancy of the Nur77 and Nor-1 orphan steroid receptors in T-cell apoptosis. Embo J 16, 1865-75 (1997).
17. Liu, Z. G., Smith, S. W., McLaughlin, K. A., Schwartz, L. M. & Osborne, B. A. Apoptotic signals delivered through the T-cell receptor of a T-cell hybrid require the immediate-early gene nur77. Nature 367, 281-4 (1994).
18. Kim, S. O., Ono, K., Tobias, P. S. & Han, J. Orphan nuclear receptor Nur77 is involved in caspase-independent macrophage cell death. J Exp Med 197, 1441-52 (2003).
19. Kuang, A. A., Cado, D. & Winoto, A. Nur77 transcription activity correlates with its apoptotic function in vivo. Eur J Immunol 29, 3722-8 (1999).
20. Brenner, C. & Kroemer, G. Apoptosis. Mitochondria—the death signal integrators. Science 289, 1150-1 (2000).
21. Li, H. et al. Cytochrome c release and apoptosis induced by mitochondrial targeting of nuclear orphan receptor TR3. Science 289, 1159-64 (2000).
22. Nopora, A. & Brocker, T. Bcl-2 controls dendritic cell longevity in vivo. J Immunol 169, 3006-14 (2002).
23. Pirtskhalaishvili, G. et al. Transduction of dendritic cells with Bcl-xL increases their resistance to prostate cancer-induced apoptosis and antitumor effect in mice. J Immunol 165, 1956-64 (2000).
24. Sotomayor, E. M., Borrello, I., Tubb, E., Allison, J. P. & Levitsky, H. I. In vivo blockade of CTLA-4 enhances the priming of responsive T cells but fails to prevent the induction of tumor antigen-specific tolerance. Proc Natl Acad Sci USA 96, 11476-81 (1999).
25. Staveley-O'Carroll, K. et al. Induction of antigen-specific T cell energy: An early event in the course of tumor progression. Proc Natl Acad Sci USA 95, 1178-83 (1998).
26. Adler, A. J. et al. CD4+ T cell tolerance to parenchymal self-antigens requires presentation by bone marrow-derived antigen-presenting cells. J Exp Med 187, 1555-64 (1998).
27. Dalyot-Herman, N., Bathe, O. F. & Malek, T. R. Reversal of CD8+ T cell ignorance and induction of anti-tumor immunity by peptide-pulsed APC. J Immunol 165, 6731-7 (2000).
28. Strome, S. E. et al. Strategies for antigen loading of dendritic cells to enhance the antitumor immune response. Cancer Res 62, 1884-9 (2002).
29. Schnurr, M. et al. Apoptotic Pancreatic Tumor Cells Are Superior to Cell Lysates in Promoting Cross-Priming of Cytotoxic T Cells and Activate NK and gammadelta T Cells. Cancer Res 62, 2347-52 (2002).
30. Lambert, L. A., Gibson, G. R., Maloney, M. & Barth, R. J., Jr. Equipotent generation of protective antitumor immunity by various methods of dendritic cell loading with whole cell tumor antigens. J Immunother 24, 232-6 (2001).
31. Asavaroengchai, W., Kotera, Y. & Mule, J. J. Tumor lysate-pulsed dendritic cells can elicit an effective antitumor immune response during early lymphoid recovery. Proc Natl Acad Sci USA 99, 931-6 (2002).
32. Parajuli, P. et al. Flt3 ligand and granulocyte-macrophage colony-stimulating factor preferentially expand and stimulate different dendritic and T-cell subsets. Exp Hematol 29, 1185-93 (2001).
33. Fong, L. et al. Altered peptide ligand vaccination with Flt3 ligand expanded dendritic cells for tumor immunotherapy. Proc Natl Acad Sci USA 98, 8809-14 (2001).
34. Gilliet, M. et al. The development of murine plasmacytoid dendritic cell precursors is differentially regulated by FLT3-ligand and granulocyte/macrophage colony-stimulating factor. J Exp Med 195, 953-8 (2002).
35. Diehl, L. et al. CD40 activation in vivo overcomes peptide-induced peripheral cytotoxic T-lymphocyte tolerance and augments anti-tumor vaccine efficacy. Nat Med 5, 774-9 (1999).
36. Sotomayor, E. M. et al. Conversion of tumor-specific CD4+ T-cell tolerance to T-cell priming through in vivo ligation of CD40. Nat Med 5, 780-7 (1999).
37. Cui, Y. et al. Immunotherapy of established tumors using bone marrow transplantation with antigen gene-modified hematopoietic stem cells. Nat Med 9, 952-958 (2003).
38. Guerder, S. & Matzinger, P. A fail-safe mechanism for maintaining self-tolerance. J Exp Med 176, 553-64 (1992).
39. Sparwasser, T. et al. Bacterial DNA and immunostimulatory CpG oligonucleotides trigger maturation and activation of murine dendritic cells. Eur J Immunol 28, 2045-54 (1998).
40. Bauer, M. et al. Bacterial CpG-DNA triggers activation and maturation of human CD11c−, CD123+ dendritic cells. J Immunol 166, 5000-7 (2001).
41. Kadowaki, N., Antonenko, S. & Liu, Y. J. Distinct CpG DNA and polyinosinic-polycytidylic acid double-stranded RNA, respectively, stimulate CD11c− type 2 dendritic cell precursors and CD11c+ dendritic cells to produce type I IFN. J Immunol 166, 2291-5 (2001).
42. Park, Y., Lee, S. W. & Sung, Y. C. Cutting Edge: CpG DNA inhibits dendritic cell apoptosis by up-regulating cellular inhibitor of apoptosis proteins through the phosphatidylinositide-3′-OH kinase pathway. J Immunol 168, 5-8 (2002).
43. Merad, M., Sugie, T., Engleman, E. G. & Fong, L. In vivo manipulation of dendritic cells to induce therapeutic immunity. Blood 99, 1676-82 (2002).
44. Doxsee, C. L. et al. The immune response modifier and Toll-like receptor 7 agonist S-27609 selectively induces IL-12 and TNF-alpha production in CD11c+CD11b+CD8− dendritic cells. J Immunol 171, 1156-63 (2003).
45. Gibson, S. J. et al. Plasmacytoid dendritic cells produce cytokines and mature in response to the TLR7 agonists, imiquimod and resiquimod. Cell Immunol 218, 74-86 (2002).
46. Nakano, H., Yanagita, M. & Gunn, M. D. CD11c(+)B220(+)Gr-1(+) cells in mouse lymph nodes and spleen display characteristics of plasmacytoid dendritic cells. J Exp Med 194, 1171-8 (2001).
47. Shen, Z., Reznikoff, G., Dranoff, G. & Rock, K. L. Cloned dendritic cells can present exogenous antigens on both MHC class I and class II molecules. J Immunol 158, 2723-30 (1997).
48. Langenkamp, A., Messi, M., Lanzavecchia, A. & Sallusto, F. Kinetics of dendritic cell activation: impact on priming of TH1, TH2 and nonpolarized T cells. Nat Immunol 1, 311-6 (2000).
49. Matzinger, P. The JAM test. A simple assay for DNA fragmentation and cell death. J Immunol Methods 145, 185-92 (1991).
50. Borrello, I. et al. Sustaining the graft-versus-tumor effect through posttransplant immunization with granulocyte-macrophage colony-stimulating factor (GM-CSF)producing tumor vaccines. Blood 95, 3011-9 (2000).

What is claimed is:

1. A method for inhibiting apoptosis in dendritic cells comprising the administration to the dendritic cells an agent which prevents or inhibits the expression of the MINOR gene in said dendritic cells.

2. A method of claim 1 wherein said agent is administered to the dendritic cells ex vivo.

3. A method of claim 1 wherein said agent is administered to the dendritic cells in vivo.

4. A method of claim 1 wherein said agent is a small interfering RNA.

5. A method of claim 1 wherein said agent is an anti-sense nucleotide molecule.

6. A method of claim 4 wherein the small interfering RNA is comprised of the double stranded nucleotide sequence of 5′ GATCCCCTGCCCTTGTCCGAGCTrTATTCAAGAGATAAAGCTCGGACAAGGGCATTTTTGGAAA-3′ (SEQ ID NO: 2); forward and 5′AGCTTTTCCAAAAATGCCCTTGTCCGAGCTTTATCTCTTGAATAAAGCTCGGACAAGGGCAGGG-3′(SEQ ID NO: 3); reverse.

7. A method of claim 1 wherein said agent inhibits signal transduction leading to the expression of MINOR.

8. A method of claim 1 wherein said agent inhibits the intracellular activity of MINOR.

9. The method of claim 1, wherein the expression of MINOR in the dendritic cells is decreased by transduction of the cells with a lentiviral vector encoding an siRNA construct having substantial sequence homology to MINOR.

10. A method for decreasing the expression of a protein in a cell population, said method comprising the steps of generating a lentiviral vector encoding an siRNA construct having substantial sequence homology to said protein, and transducing the cell population with said lentiviral vector.

11. The method of claim 10, wherein the protein is MINOR.

12. The method of claim 1, wherein the dendritic cells are bone marrow dendritic cells.

13. A method for increasing the survival time of ex vivo-generated dendritic cells following infusion of said cells into a subject, the method comprising the ex vivo transduction of said cells with a lentiviral vector encoding an siRNA construct having substantial sequence homology to MINOR and infusing the transduced cells into the subject.

14. The method of claim 13, wherein the subject is human.

15. The method of claim 13, wherein the dendritic cells are bone marrow dendritic cells.

16. A method for enhancing the antigen presenting ability of dendritic cells, said method comprising transducing said cells with a lentiviral vector encoding an siRNA construct having substantial sequence homology to MINOR.

17. The method of claim 16, wherein the dendritic cells are bone marrow dendritic cells.

18. A method for enhancing the capacity for dendritic cells to stimulate tolerant T cells, said method comprising transducing said cells with a lentiviral vector encoding an siRNA construct having substantial sequence homology to MINOR.

19. The method of claim 18, wherein the dendritic cells are bone marrow dendritic cells.

20. A dendritic cell-based vaccine comprising dendritic cells expressing siRNA's having substantial sequence homology to MINOR.

21. The vaccine of claim 20, wherein said vaccine is for cancer, viral disease, bacterial disease, or immune disorders.

22. The vaccine of claim 21, wherein said vaccine is for cancer.

23. A method for preparing the vaccine of claim 20, comprising the step of preparing an siRNA construct having substantial sequence homology to MINOR and ex vivo transducing dendritic cells with said construct.

24. A method of preserving the CD11c+ population of dendritic cells, comprising transducing hematopoietic stem-progenitor cells with a lentiviral vector encoding an siRNA construct having substantial sequence homology to MINOR.

25. A method for stably decreasing or substantially suppressing the expression of MINOR in dendritic cells, said method comprising the steps of transducing hematopoietic stem-progenitor cells with a lentiviral vector encoding an siRNA construct having substantial sequence homology to MINOR and transplanting the transduced cells into a myeloablatively treated mammalian subject.

26. The method of claim 20, wherein the mammalian subject is human.

27. A method of augmenting an immune response specific for an antigen in an individual, comprising the steps of: (a) obtaining dendritic cells from the individual; (b) causing the dendritic cells to express the antigen by either (i) exposing the dendritic cells to the antigen in culture under conditions promoting uptake and processing of the antigen, or (ii) transfecting the dendritic cells with a gene encoding the antigen; (c) activating the antigen-expressing dendritic cells, (d) treating the dendritic cells with an agent that inhibits MINOR expression; and. (e) administering the activated, antigen-expressing dendritic cells to the individual.

28. The method of claim 27, wherein the dendritic cells are obtained by obtaining hematopoietic stem or progenitor cells from the individual, and contacting the hematopoietic stem or progenitor cells with an agent selected from the group consisting of flt-3 ligand, GM-CSF, IL-4, TNF-.alpha., IL-3, c-kit ligand, fusions of GM-CSF and IL-3, and combinations thereof.

29. The method of claim 27 wherein the agent inhibiting MINOR expression is a small interfering RNA.

30. The method of claim 27 wherein the agent inhibits signal transduction in dendritic cells resulting in the expression of MINOR.

31. The method of claim 27 wherein the agent inhibits the intracellular activity of MINOR.

32. A small interfering RNA comprising the double stranded nucleotide sequence of 5′GATCCCCTGCCCTTGTCCGAGCTTTATTCAAGAGATAAAGCTCGGACAAGGGCATTTTTGGAAA-3′; forward and 5′AGCTTTTCCAAAAATGCCCTTGTCCGAGCTTTATCTCTTGAATAAAGCTCGGACAAGGGCAGGG-3′; reverse.

33. A dendritic cell expressing the small interfering RNA of claim 32.

34. A population of dendritic cells for use in vaccination of a subject produced by the process of (a) obtaining dendritic cells from the individual; (b) causing the dendritic cells to express the antigen by either (i) exposing the dendritic cells to the antigen in culture under conditions promoting uptake and processing of the antigen, or (ii) transfecting the dendritic cells with a gene encoding the antigen; (c) activating the antigen-expressing dendritic cells, (d) treating the dendritic cells with an agent that inhibits MINOR expression.

35. A population of dendritic cells of claim 34 wherein the agent that inhibits MINOR &expression is a nucleotide construct containing a small interfering RNA.

36. A population of dendritic cells of claim 35 wherein the small interfering RNA is comprised the double stranded nucleotide sequence of 5′GATCCCCTGCCCTTGTCCGAGCTTTATTCAAGAGATAAAGCTCGGACAAGGGCATTTTTGGAAA-3′; forward and 5′AGCTTTTCCAAAAATGCCCTTGTCCGAGCTTTATCTCTTGAATAAAGCTCGGACAAGGGCAGGG-3′; reverse.
